(19) United States
(12) Patent Application Publication
(10) Pub. No.: US 2026/0273518 A1
(43) Pub. Date: Sep. 17, 2026

(54) BULKY AND SMALL-STERIC-HINDRANCE LIGAND AND USE THEREOF

(71) Applicants: Dagang Li, Nanjing (CN); Song Li, Beijing (CN); DG HC OPTICAL CO., LTD, Dongguan (CN)

(72) Inventors: Dagang Li, Nanjing (CN); Song Li, Beijing (CN)

(21) Appl. No.: 19/448,138

(22) Filed: Jan. 14, 2026

(30) Foreign Application Priority Data
Mar. 6, 2025 (CN) 202510262791.3

(51) Int. Cl. B01J 31/24; B01J 31/14; B01J 31/18; C07D 207/06; C07D 211/12; C07D 241/04; C07D 265/30; C07D 307/06; C07D 309/04; C07D 333/10; C07D 335/02; C07D 453/02; C07D 455/04; C07F 5/06; C07F 9/6568; C07F 9/6584; C07F 9/6596

(52) U.S. Cl. CPC B01J 31/2423; B01J 31/143; B01J 31/181; B01J 31/2409; B01J 31/2495; C07D 207/06; C07D 211/12; C07D 241/04; C07D 265/30; C07D 307/06; C07D 309/04; C07D 333/10; C07D 335/02; C07D 453/02; C07D 455/04; C07F 5/062; C07F 9/65683; C07F 9/6584; C07F 9/6596; B01J 2231/20; B01J 2531/004; B01J 2531/62

(57) ABSTRACT

Provided are a bulky and small-steric-hindrance ligand and use thereof, belonging to the field of chemical catalytic technology. It is a ligand for a catalytic system used in the selective oligomerization of ethylene, which produces 1-octene as the main product and 1-hexene and 1-butene as by-products. The ligand is a ternary catalytic system composed of a central metal chromium, a ligand, and an aluminum-containing activator. During the in-situ preparation process, the ligand can regulate the molecular self-assembly in the process of formation of catalytically active species. This achieves selective oligomerization of ethylene that exclusively produces three useful comonomers: 1-octene as the main product and 1-hexene and 1-butene as byproducts. The distribution of the three monomers is adjustable without generating any olefin waste. For polyolefin enterprises, only one set of oligomerization equipment using this catalyst needs to be constructed to meet the demand for the three different copolymerization monomers.

### CROSS-REFERENCE TO RELATED APPLICATION

The present application claims priority from Chinese Patent Application No. 202510262791.3 filed on Mar. 6, 2025, the contents of which are incorporated herein by reference in their entirety.

### FIELD OF THE INVENTION

The present disclosure belongs to the technical field of chemical catalysis, and particularly relates to a bulky and small-steric-hindrance ligand and use thereof.

### BACKGROUND OF THE INVENTION

1-Octene is an excellent comonomer for polyolefins and an important raw material for POE elastomers. Conventional technologies produce full-range linear olefins with 4-40 carbon atoms, but the selectivity for 1-octene is extremely low. Sasol Technology Co., Ltd. filed Chinese Patent Application CN200580025945.8 (International Publication No. WO2005123633A1, published on Dec. 29, 2005) on Jun. 13, 2005. This application modified the ligand (MeOPh)₂PN(Me)P(MeOPh)₂ invented by D. F. Wass of BP by removing one MeO group to form the (Ph)₂PN(iPr)P(Ph)₂ ligand. When activated with MMAO-3A (Methylaluminoxane-3A) together with a trivalent chromium source, the catalyst, originally designed for the selective trimerization of ethylene to 1-hexene, is transformed into a catalyst for the selective tetramerization of ethylene to 1-octene. The main drawbacks of the existing Sasol tetramerization catalyst are as follows: 1) Low selectivity for the main product 1-octene, only about 70%; 2) Low ortho-terminal olefin content of the secondary product hexene, around 80%; 3) Approximately 15% of ethylene is converted into non-target products; 4) High dosage and cost of the activator, with an Al/Cr ratio exceeding 500.

To address these drawbacks, numerous studies have been conducted, but the upper limit of around 70% selectivity for 1-octene has not been broken. It was not until 2012 that R. Duthateau, Y. Shaikh, et al. achieved a 1-octene selectivity of 91% using a PNR(CH₂)₃RNP-type ligand and CrCl₂(THF)₂ as the chromium source, activated with DMAO/R₃Al. However, the catalytic activity was extremely low, at less than 2.0 kg/gCr·h. When CrCl₃(THF)₃ was used as the chromium source, the 1-octene selectivity decreased to 81% (with 19% for 1-hexene), but the activity remained low at only 25.7 kg/gCr·h, rendering it practically useless. Over the past five years, improving the catalytic activity of the catalyst developed by R. Duthateau's team has become a research focus, with many research groups engaging in the study of ethylene oligomerization catalysts. However, as shown in Table 1, the selectivity for 1-octene has hovered around 70%.

TABLE 1
Novel Catalysts for Ethylene Tetramerization to 1-Octene Reported in Recent Years

| Serial | | | | | | | Catalytic | |
|---|---|---|---|---|---|---|---|---|
| No. | Inventor | Patent No. | Applicant | C6 | C8 | PE | Activity | |
| 1 | Li | CN201710096548.4 | Li Song | 15-20 | 70-80 | — | 80-150 | |
| | Dagang | | | | | | kg/gcr · h | |
| 2 | M. J. Shin | US2019/0224658A | KR | 45 | 50 | 4.4 | 3.1 | kg/mmolCr |
| 3 | J. Klosin | US2018/0291048A1 | Dow | 17.8 | 77.0 | 0.45 | 13.9 | t/gh |
| 4 | Tao Jiang | US2019/0106366A1 | Tianjin | 13.8 | 71.1 | — | 119 | kg/gCrh |
| | | | University | | | | | |
| | | | of Science | | | | | |
| | | | and | | | | | |
| | | | Technology | | | | | |
| 5 | U. Kilgo | CN201880077347.2 | CP | 35 | 57 | 0.88 | 229 | kg/gCr h |
| 6 | Chen | CN202010342323.4 | Yantai | — | 79 | 0.8 | 485 | kg/gCr h |

| | |
|---|---|
| Guanliang | Wanhua |
| et al. | |

In summary, despite various follow-up improvements over the past 15 years, the overall performance indicators have not yet exceeded those of the catalyst first discovered by Sasol. Consequently, for its industrial plant (planned in 2014) with an annual output of 100,000 tons of 1-octene and hexene via ethylene oligomerization, Sasol had to adopt the originally discovered (Ph)₂PN(iPr)P(Ph)₂/Cr/MMAO catalytic system. Therefore, the present disclosure is directed to the innovative research of catalysts for the selective oligomerization of ethylene.

It is well known to those skilled in the art that the selective trimerization of ethylene to 1-hexene and the selective tetramerization of ethylene to 1-octene follow the same reaction mechanism. Given the identical mechanism, why can the target product 1-hexene achieve a laboratory selectivity of 100% and an industrial selectivity of 92-96% in chromium-based catalytic systems incorporating halides as the fourth component, while the selectivity for 1-octene in ethylene tetramerization has failed to break through the ceiling of around 70%.

### SUMMARY OF THE INVENTION

To address the shortcomings of the prior art, the present application provides a bulky and small-steric-hindrance ligand and use thereof, solving technical problems such as insufficient basic theoretical understanding of ethylene oligomerization and defects in practical applications in the prior art.

The present disclosure adopts the following technical solutions:

A bulky and small-steric-hindrance ligand, wherein the bulky and small-steric-hindrance ligand is a monodentate bulky and small-steric-hindrance ligand constructed from dentate groups consisting of dentate elements without substituents, a monodentate bulky and small-steric-hindrance ligand constructed from dentate groups consisting of dentate elements containing branched substituents, a bidentate ligand composed of two dentate groups of P or N linked by a bridging group, or a bidentate ligand composed of two identical or different dentate groups containing one or more dentate elements selected from P, N, S, and O linked by a bridging group that does not change configuration due to σ-rotation, the bridging group has a double-bonded conjugated system; the dentate elements are one or more of P, N, S, and O.

Preferably, the monodentate bulky and small-steric-hindrance ligand constructed from dentate groups consisting of dentate elements containing branched substituents is specifically one or more of the following: a monodentate tertiary phosphine ligand compound containing only one dentate group, a monodentate tertiary amine ligand compound containing only one dentate group, a monodentate thioheterocyclic ligand compound containing only one dentate group, and a monodentate oxoheterocyclic ligand compounds containing only one dentate group; the bidentate ligand composed of two identical or different dentate groups containing one or more dentate elements selected from P, N, S, and O linked by a bridging group that does not change configuration due too-rotation is a bidentate ligand compound composed of two dentate groups linked by a bridging group, wherein the two dentate groups of the bidentate ligand are located at two coordination sites that can form a straight line with the central metal, and the ligand and the central metal form an arch-shaped structure, providing sufficient operational space for the self-assembly of the four n-ethylene groups located at the central metal into the target product 1-octene.

Preferably, the monodentate bulky and small-steric-hindrance ligand constructed from dentate groups consisting of dentate elements without substituents is as follows:

| Serial | | Skeletal | |
|---|---|---|---|
| No. | Ligand | Formula | Substituents and Coefficients |
| 1 | 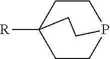 | C₇H₁₂P | R = Cph₃, CEt₃, (CH₂)nCH₃, Ph(CH₂)nCH₃, C₆h₁₁(CH₂)nCH₃, PhCOOR |
| 2 | 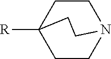 | C₇H₁₂N | R = Cph₃, CEt₃, (CH₂)nCH₃, Ph(CH₂)nCH₃, C₆H₁₁(CH₂)nCH₃ |
| 3 | 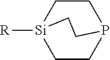 | C₆H₁₂PSi | R = Cph₃, (CH₂)nCH₃, CEt₃, Ph(CH₂)nCH₃ |
| 4 | 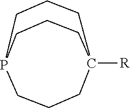 | C₁₀H₁₈P | R = Cph₃, (CH₂)nCH₃, CEt₃, Ph(CH₂)nCH₃ |
| 5 | 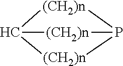 | — | n = 3, 4, 5, 6, 7 |
| 6 | 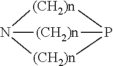 | — | n = 3, 4, 5, 6, 7 |
| 7 | 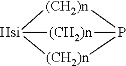 | — | n = 3, 4, 5, 6, 7 |
| 8 | 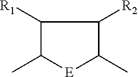 | C₁₀H₁₈ | R₁ = R₂ = Et, Pr E = S, O |
| 9 | 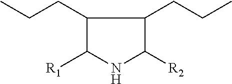 | C₁₀H₁₉N | R₁ = R₂ = H, Me |
| 10 | 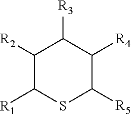 | C₅H₅S | R₁ = R₅ = H, Me R₂ = R₄ = H, Et, Pr, t-Bu R₃ = H, Pr, Cph₃, AlEt₂ |
| 11 | 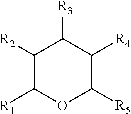 | C₅H₅O | R₁ = R₅ = H, Me R₂ = R₄ = H, Et, Pr, t-Bu R₃ = H, Pr, t-Bu, Cph₃ |
| 12 | 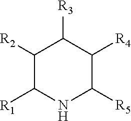 | C₅H₆N | R₁ = R₅ = H, Me R₂ = R₄ = H, Et, Pr, t-Bu R₃ = H, Pr, t-Bu, Cph₃ |
| 13 | 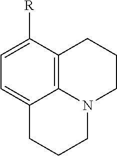 | C₁₂H₁₄N | R = Cph₃, CEt₃, Cpr₃, CMe₃ |
| 14 | 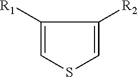 | C₄H₂S | R₁ = R₂ = Cph₃, CMe₃, CEt₃, Cpr₃, AlEt₂, Al(iBu)₂ |
| 15 | 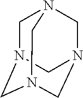 | C₆H₁₂N₄ | — |
| 16 | 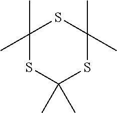 | C₉H₁₈S₃ | — |
| 17 | 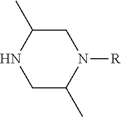 | C₆H₁₃N₂ | R = cph₃, CEt₃, AlEt₂, Al(iBu)₂, C(t-Bu)₃ |
| 18 | 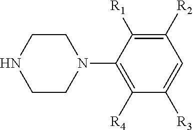 | C₁₁H₁₂N₂ | R₁ = R₄ = H, Me R₃ = R₂ = Pr t-Bu |
| 19 | 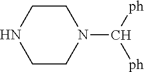 | C₁₇H₂₀N₂ | — |
| 20 | 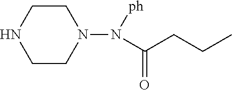 | C₁₄H₂₀N₂O | — |
| 21 | 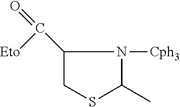 | C₅H₇O₂NS | — |
| 22 | 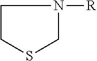 | C₃H₆NS | R = AlEt₂, Ali-Bu₃, cph₃, cpr₃ |
| 23 | 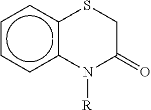 | C₈H₆NOS | R = cph₃, CEt₃, cpr₃, C(t-Bu)₃ |
| 24 | 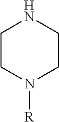 | C₅H₁₁N₂ | R = cph₃, CEt₃, AlEt₂, Al(i-Bu)₂ |
| 25 | 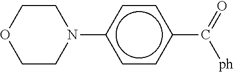 | C₁₇H₁₇NO₂ | — |
| 26 | 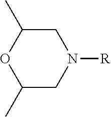 | C₆H₁₂NO | R = cph₃, CEt₃, Cpr₃, C(t-Bu)₃, Al(Bu)₃ |
| 27 | 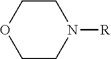 | C₄H₈NO | R = cph₃, CEt₃, Cpr₃, C(t-Bu)₃, Al(Bu)₃ |
| 28 | 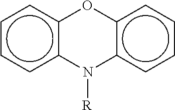 | C₁₂H₈NO | R = cph₃, CEt₃, AlEt₂, Cpr₃, C(t-Bu)₃, Al(i-Bu)₂ |
| 29 | 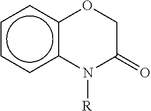 | C₈H₆NO₂ | R = cph₃, CEt₃, AlEt₂, Cpr₃, C(t-Bu)₃, Al(i-Bu)₂ |
| 30 | 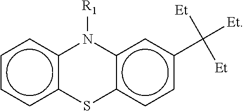 | C₁₉H₂₂NS | R = cph₃, AlEt₂, CEt₃ |

Preferably, the monodentate bulky and small-steric-hindrance ligand constructed from dentate groups consisting of dentate elements containing branched substituents is as follows:

| Serial | | Skeletal | | | | |
|---|---|---|---|---|---|---|
| No. | Ligand | Formula | Substituents and Coefficients | | | |
| 31 | 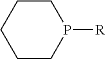 | PC₅H₁₀ | 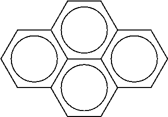 | 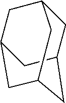 | 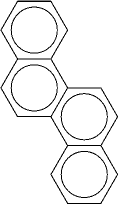 | 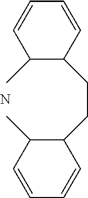 |
| 32 | 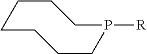 | PC₇H₁₄ | 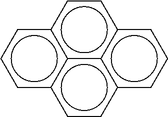 | 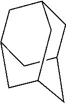 | 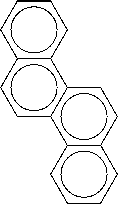 | 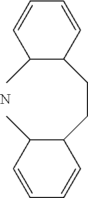 |
| 33 | 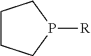 | PC₄H₈ | 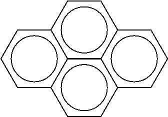 | 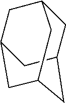 | 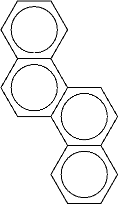 | 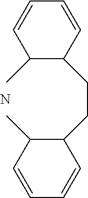 |
| 34 | 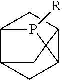 | PC₇H₁₀ | 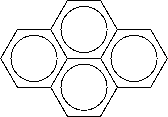 | 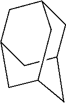 | 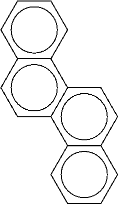 | 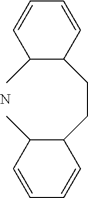 |
| 35 | 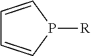 | PC₄H₄ | 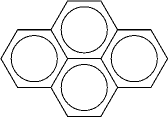 | 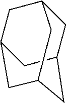 | 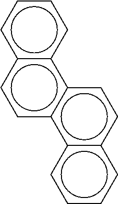 | 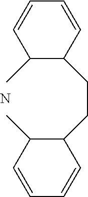 |
| 36 | 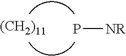 | PC₁₁H₂₂ | NR = 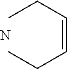 | 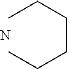 | 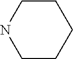 | 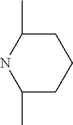 |

| | | |
|---|---|---|
| 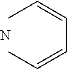 | 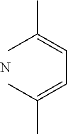 | 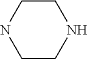 |

| | | | |
|---|---|---|---|
| 37 | 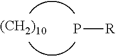 | PC₁₀H₂₀ | R = Me, Et, Pr, ph, C₄H₁₁ = 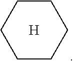 |

Preferably, the bidentate ligand composed of two dentate groups of P or N linked by a bridging group is:

| Seri- | | | | | |
|---|---|---|---|---|---|
| al | | | Dental Group and | | |
| No. | Ligand | Skeletal Formula | Coefficient | | |
| 38 | CP₁—(CH₂)n—CP₂ | —(CH₂)n— | cp₁ = 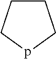 | 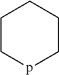 | pph₂ |

| | |
|---|---|
| cp₂ = 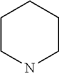 | 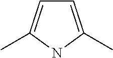 |

| | | | n = 3, 5, 7, 9, 4 | |
|---|---|---|---|---|
| | | | cp₁ = cp₂ or cp₁ ≠ cp₂ | |
| 39 | CP—N(Me)—(CH₂)ₙ—N(Me)—CP | —(Me)N—(CH₂)ₙN(Me)— | CP = 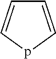 | 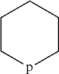 |

| | | | 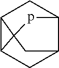 | |
|---|---|---|---|---|
| | | | pph₂ 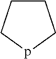 | C₇H₁₀P |
| | | | n = 3, 4, 5, 6, 7 | |
| 40 | CPNH(CH₂)O(CH₂)O(CH₂)NHCP | C₈H₁₈N₂O₂ | CP = 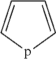 | 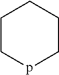 |
| | | | 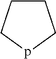 | |
| | | | 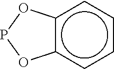 | |
| | | | 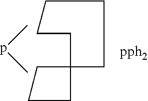 | |
| 41 | 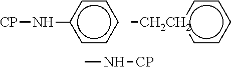 | C₁₄H₁₄N₂ | CP = 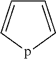 | 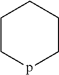 |
| | | | 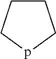 | |
| | | | 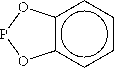 | |
| | | | 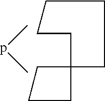 | pph₂ |
| 42 | CP—NH—(CH₂)₃—NH—(CH₂)₃—NH—CP | C₆H₁₅N₃ | CP = 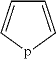 | 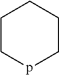 |
| | | | 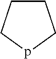 | |
| | | | 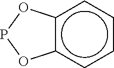 | |
| | | | 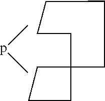 | pph_{2.} |

Preferably, the bidentate ligand composed of two identical or different dentate groups containing one or more dentate elements selected from P, N, S, and O linked by a bridging group that does not change configuration due to σ-rotation is:

| Se- | | | | | |
|---|---|---|---|---|---|
| ri- | | | | | |
| al | | Skeletal | | | |
| No. | Ligand | Formula | Dental Group and Coefficient | | |
| 43 | 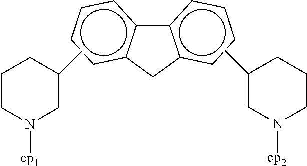 | C₂₃H₂₆N₂ | cp₁ = cp₂ 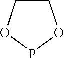 pph₂ | 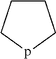 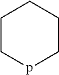 | 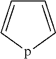 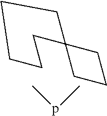 |
| 44 | 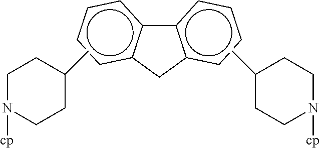 | C₂₃N₂₆N₂ | cp₁ = cp₂ 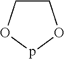 pph₂ | 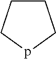 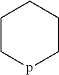 | 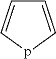 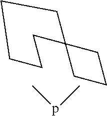 |
| 45 | 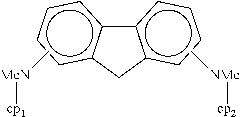 | C₁₅H₁₄N₂ | cp₁ = cp₂ 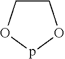 pph₂ | 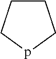 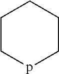 | 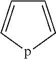 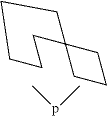 |
| 46 | 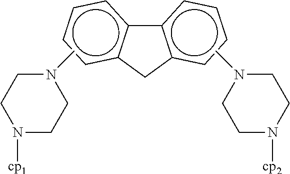 | C₂₁H₂₄N₄ | cp₁ = cp₂ 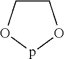 pph₂ | 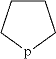 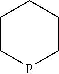 | 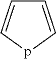 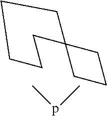 |
| 47 | 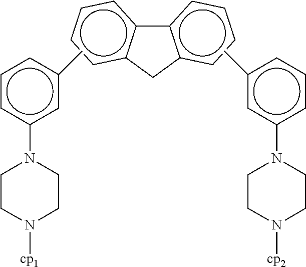 | C₃₃H₃₂N₄ | cp₁ = cp₂ 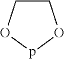 | 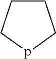 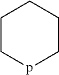 | 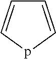 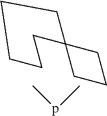 |

| | | | | | |
|---|---|---|---|---|---|
| 48 | 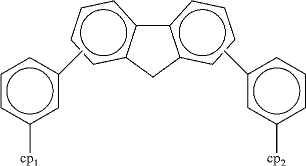 | C₂₅H₁₅ | c₁ ≠ cp₂ cp₁ = cp_{2 =} 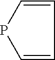 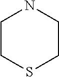 | 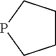 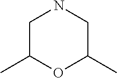 | 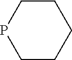 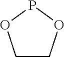 |
| | | | pph₂ | 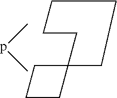 | 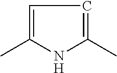 |
| | | | 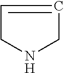 | 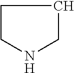 | 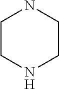 |
| 49 | 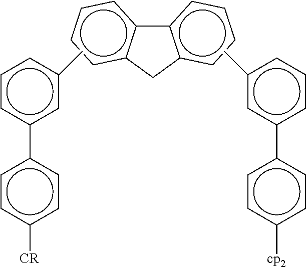 | C₃₇H₂₄ | c₁ ≠ cp₂ cp₁ = cp₂ = 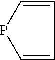 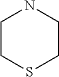 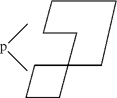 | 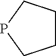 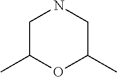 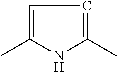 | 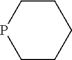 pph₂ 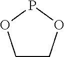 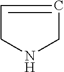 |

| | |
|---|---|
| 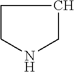 | 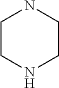 |

| 50 | 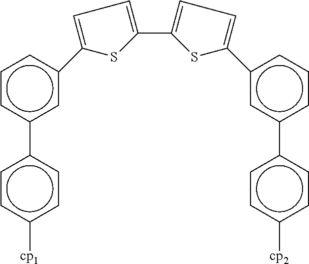 | C₃₂H₂₀S₂ | cp₁ ≠ cp [symbol] cp [symbol] = cp [symbol] = 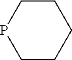 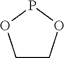 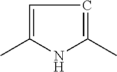 | 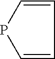 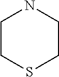 pph₂ | 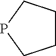 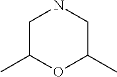 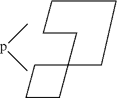 |
|---|---|---|---|---|---|
| | | | 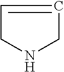 | 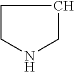 | 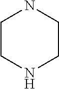 |
| 51 | 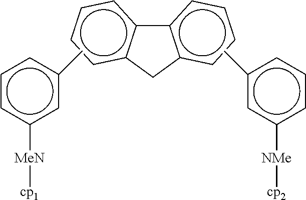 | C₂₇H₂₂N₂ | 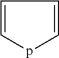 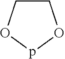 | 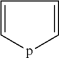 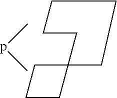 | 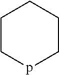 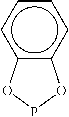 |
| | | | pph2 | | |
| 52 | 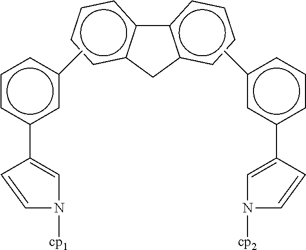 | C₃₃H₁₃N₂ | cp₁ = cp₂ 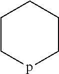 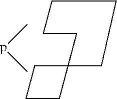 | 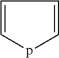 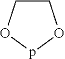 pph₂ | 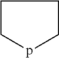 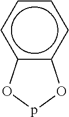 |
| | | | | 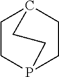 | |
| 53 | 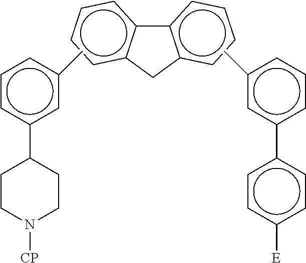 | C₃₁H₂₉N | cp = 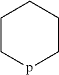 | 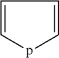 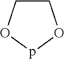 | pph₂ |
| | | | E = 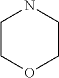 | 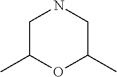 | 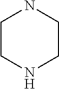 |
| | | | 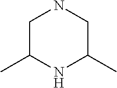 | 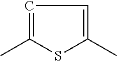 | 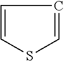 |
| | | | 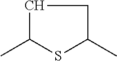 | 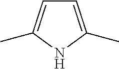 | |
| 54 | 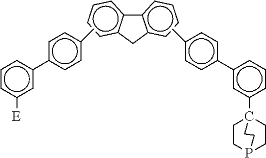 | C₄₄H₃₆P | E = 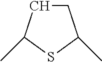 | 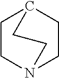 | 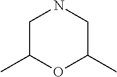 |
| 55 | 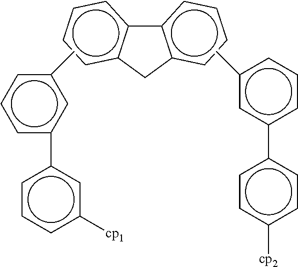 | C₃₇H₂₄ | cp₁ = cp₂ = 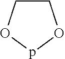 | 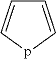 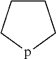 | 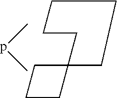 pph₂ |
| 56 | 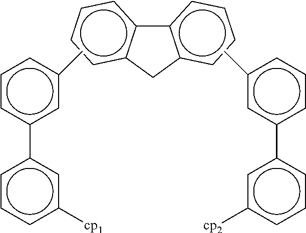 | C₃₇H₂₄ | cp₁ = cp₂ = 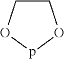 | 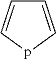 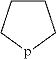 | 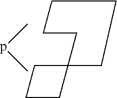 pph₂ |

| | | | | |
|---|---|---|---|---|
| 57 | 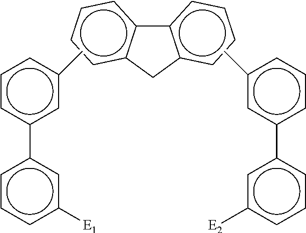 | C₃₇H₂₄ | E₁ = E₂ or 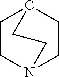 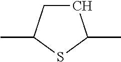 | E₁ ≠ E₂ 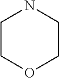 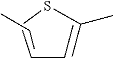 |

| | | | |
|---|---|---|---|
| 58 | 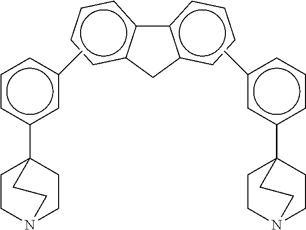 | C₂₇H₃₃N₂ | — |
| 59 | 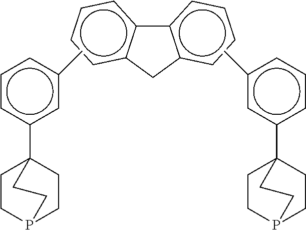 | C₂₇H₃₃P₂ | — |
| 60 | 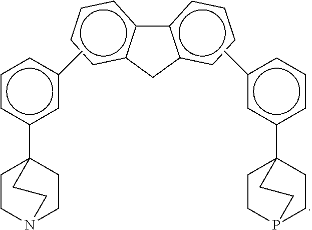 | C₂₇H₃₃NP | — |

[symbol]

indicates data missing or illegible when filed

the bridging group has a double-bonded conjugated system.

Preferably, the dentate groups are specifically as follows:
Dentate group 1, molecular formula: PPh₂;
Dentate group 2, molecular formula: C₈H₁₄P, structural formula:

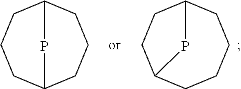

Dentate group 3, molecular formula: PC₄H₈;
Dentate group 4, molecular formula: PC₅H₁₀, structural formula:

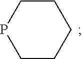

Dentate group 5, molecular formula: PC₆H₁₀O₂, structural formula:

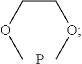

Dentate group 6, molecular formula: PC₇H₁₂, structural formula:

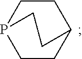

Dentate group 7, molecular formula: NC₇H₁₂, structural formula:

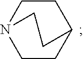

Dentate group 8, molecular formula: NC₄H₈S, structural formula:

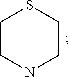

Dentate group 9, molecular formula: NC₄H₄;
Dentate group 10, molecular formula: NC₄H₆;
Dentate group 11, molecular formula: N₂C₄H₉, structural formula:

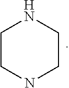

Use of the bulky and small-steric-hindrance ligand describe above in a ternary catalytic system, wherein the ternary catalytic system is a ligand/transition metal/activator ternary catalytic system composed of a bulky and small-steric-hindrance ligand, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

Preferably, the activator is an aluminum-containing activator, and the ternary catalytic system catalyzes the selective oligomerization of ethylene to produce three linear normal α-olefin products, generating only three linear normal α-olefin products of 1-octene, 1-hexene, and 1-butene, without generating C10-C20 waste olefins, and the carbon utilization rate is 100%.

Preferably, the aluminum-containing activator is an alkylaluminum compound and/or an alkoxyaluminum compound, the alkylaluminum compound is specifically one or more of triethylaluminum, trimethylaluminum, and triisobutylaluminum, the alkoxyaluminum compound is specifically one or more of MAO, MMAO, DMAO, and MMAO-3A.

Principle: The bulky and small-steric-hindrance ligand disclosed in the present application functions as follows: the bulky ligand regulates the self-assembly of the catalyst, confining the two dentate groups of the ligand to coordinate only to two coordination sites linearly aligned with the central metal. It also modulates four n-ethylene molecules to coordinate to four coordination sites of the central chromium, which can form a planar transient conformation, thereby constructing a novel catalytic model. The dentate groups with small steric hindrance exert minimal interference during the molecular self-assembly process of n-ethylene conversion into target products. Therefore, the bulky and small-steric-hindrance ligand, together with the central metal and activator, constitutes a ternary catalytic system of ligand/transition metal/activator for catalyzing ethylene oligomerization. The resulting α-olefin products exhibit three key characteristics: 1) Only three types of olefins are generated, with C8 olefin as the main product and C6/C4 olefins as by-products, and their product distribution is adjustable; 2) High linear terminal olefin content: 1-butene>99%, 1-hexene>98%, and 1-octene>97%; 3) No C10-C20 higher olefins are produced, with carbon utilization rate approaching 100%.

### Beneficial Effects

1. For ten-million-ton-scale polyolefin production enterprises, the wide range of polyolefin product grades requires various comonomers. Purchasing comonomers externally at high costs reduces the competitiveness of the enterprise's own brands. With the present disclosure, if the enterprise produces comonomers in-house, it only needs to construct one set of oligomerization equipment using this catalyst or replace the catalyst of existing ethylene selective oligomerization equipment for single products (including dimerization, trimerization, or tetramerization equipment) with the catalyst of the present disclosure. By adjusting the distribution ratio of the three comonomers according to the technology disclosed in present disclosure, the demand for three different comonomers can be fully met. There is no need to separately construct or operate three sets of oligomerization equipment: ethylene tetramerization for octene production, ethylene trimerization for hexene production, and ethylene dimerization for butene production.
2. Only three types of olefins are generated, with C8 olefin as the main product and C6/C4 olefins as by-products, and their product distribution is adjustable.
3. High linear terminal olefin content: 1-butene>99%, 1-hexene>98%, and 1-octene>97%.
4. No C10-C20 higher olefins are produced, with carbon utilization rate approaching 100%.

### BRIEF DESCRIPTION OF THE DRAWINGS

FIG. 1 Time-dependent in-situ high-pressure infrared spectroscopy of selective trimerization of ethylene to 1-hexene in the present application;

FIG. 2 Schematic diagram of the catalytically active species with three n-ethylene molecules in angular coordination for selective trimerization of ethylene to 1-hexene in the present application;

FIG. 3 Schematic diagram of the catalyst cycle process for selective trimerization of ethylene to 1-hexene in the present application;

FIG. 4 Chromatogram of the ethylene selective oligomerization reaction solution of the chromium-based catalyst with bulky and small-steric-hindrance ligand in the present application;

FIG. 5 Schematic diagram of the arch-shaped catalytic model formed by bidentate ligand coordinating linearly to the central metal Cr;

FIG. 6 Process flow diagram of the industrial manufacturing method for the phosphabicyclic secondary phosphine compound in the present application.

### REFERENCE NUMERALS

1. Premixer, 2. Metering pump, 3. Gas-liquid intensive mixer, 4. Oxygen-free initiator feeder, 5. Saturated liquid storage tank, 6. Diaphragm pump, 7. Pipeline reactor, 8. Water bath device, 9. Gas-liquid separator, 10. Intermediate storage tank.

### DETAILED DESCRIPTION

The present disclosure will be further described in detail below with reference to embodiments. It should be understood that the following embodiments are merely illustrative and explanatory of the present disclosure and do not limit the present disclosure to these specific embodiments or limit the scope of the present disclosure in any way.

Definitions: Bulky refers to the relatively large volume of the ligand; small steric hindrance refers to the small coordination steric hindrance around the central metal. The effect of bulky and small steric hindrance is achieved through long-chain bidentate or monodentate ligands with steric hindrance forming arch-shaped cavity coordination with the central metal.

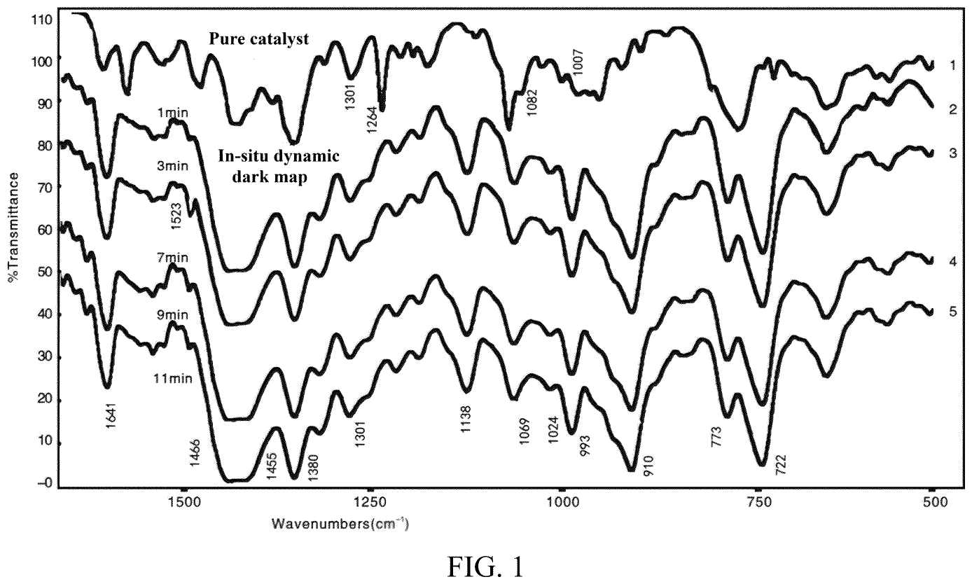

The present application conducts a comprehensive comparative study on selective trimerization of ethylene and tetramerization catalysts from macro-performance to micro-structure, and confirms the composition and structure of the catalytically active species for selective trimerization of ethylene, as shown in FIG. 1: Time-dependent in-situ high-pressure infrared spectroscopy of selective trimerization of ethylene to 1-hexene. The assignment of absorption band wavenumbers and the compounds in FIG. 1 are summarized in the Table 2 below.

TABLE 2
Summary of Wavenumbers and Assignments of Absorption Bands in In-situ
High-pressure Infrared Spectroscopy of Ethylene Trimerization Reaction

| Serial | Wavenumber | | | Time-dependent | | Assigned |
|---|---|---|---|---|---|---|
| No. | (cm⁻¹) | Category | Intensity | Change | Band Assignment | Compound |
| 1 | 722 | New peak | S | Increasing | C—H vibration of | Ethylene |
| | | | | | by-product | |
| | | | | | polyethylene | |
| 2 | 773 | Solvent | S | Unchanged | (CH₂)₄ skeleton of | Heptane |
| | | peak | | | solvent heptanes | solvent |
| 3 | 910 | New peak | S | Increasing | Out-of-plane bending of | Hexene |
| | | | | | CH₂ in product hexane | |
| 4 | 933 | New peak | S | Increasing | In-plane bending of | Hexene |
| | | | | | CH₂ in product hexane | |
| 5 | 1069 | Embedded | M | Increasing | Multiple peaks, | Multiple |
| | | peak | | | including 1082 cm⁻¹ | substances |
| | | | | | (N—Al bond stretching) | |
| 6 | 1138 | New | M | Increasing | Unknown (related to | Unknown |
| | | peak | | | ethylene) | |
| 7 | 1380 | New | S | Increasing | Antisymmetric bending | Hexene |
| | | peak | | | of CH₃ in product | |
| 8 | 1455-1466 | New | S | Increasing | CH₂ scissoring + | Multiple |
| | | broad | | | antisymmetric bending | trimerization |
| | | peak | | | of CH₃ | products |
| 9 | 1641 | New peak | M-->S | Increasing | C═C stretching | Hexene |
| | | | | | vibration of product | |
| | | | | | hexene | |
| 10 | 1264 | Disappearing | M | Disappearing | C—H bending of | Tetrachloroethane |
| | | peak | | | tetrachloroethane | |
| 11 | 1007->1024 | 1007 peak | W | Unchanged | N—Cr stretching | DMP-Cr |
| | | shift | | | vibration | |
| 12 | 1301 | Catalyst | W | Unchanged | DMP π-coordination | DMP-Cr |
| | | peak | | | | |
| 13 | 1523 | Ethylene- | W | Unchanged | Assigned to π-ethylene | C₂H₄—Cr |
| | | related | | | coordinated to Cr bond | |
| | | peak | | | | |

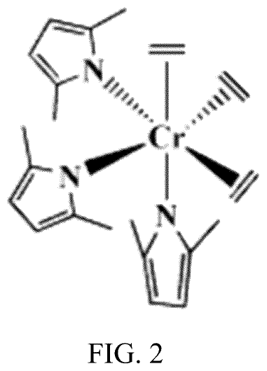

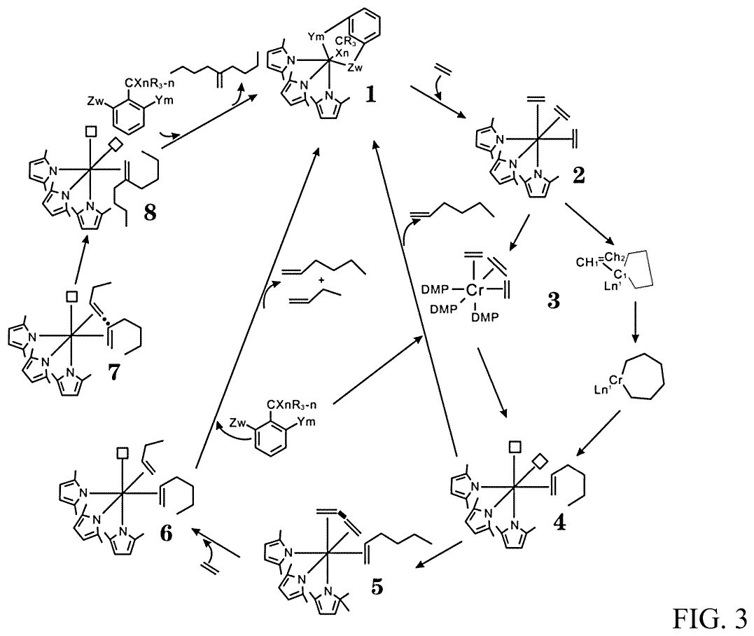

Due to the paramagnetic nature of chromium-based catalysts, which makes them difficult to characterize by in-situ high-pressure NMR, the present disclosure employs in-situ high-pressure infrared spectroscopy to explore the catalytically active species for selective trimerization of ethylene, as shown in FIG. 1. It is found that a chromium-ethylene complex exists during the selective trimerization reaction of ethylene (FIG. 2). Further studies on the selective trimerization of ethylene catalyst cycle and the modification mechanism of halides (FIG. 3) reveal that selective trimerization of ethylene to 1-hexene requires three ethylene molecules to coordinate to the central metal chromium simultaneously. Supported by catalyst molecular orbital model demonstrations and DFT calculations, it is found that selective tetramerization of ethylene also requires four π-ethylene molecules to coordinate to the central metal chromium simultaneously, and for a chromium center with a coordination number of six, these four π-ethylene molecules can form two coordination configurations. Through comprehensive comparison and summary, the prior art for selective tetramerization of ethylene shows that the two dentate groups of the ligand in the catalytic system both coordinate to adjacent coordination sites of the central metal. This configuration of the catalytically active species is the reason why it is difficult to improve the selectivity of the target product in selective tetramerization of ethylene. Based on this understanding, the innovative idea of attempting to make the two dentate groups of the tetramerization ligand self-assemble and coordinate to two coordination sites linearly aligned with the central metal naturally arises.

A series of basic studies have been conducted on the influence of halide structure on the formation of catalytically active species (FIG. 2), the catalyst cycle process (FIG. 3), and the causes of catalyst deactivation, leading to the following new insights:
(1) Selective trimerization of ethylene requires three ethylene molecules to coordinate to the central metal chromium simultaneously; selective tetramerization of ethylene requires four ethylene molecules to coordinate to the central metal chromium simultaneously.
(2) The molecular formulas of the catalytically active species for selective trimerization and tetramerization of ethylene are both (DMP)ₙCr(π-C₂H₄)₆₋ₙ, where n=number of ligand dentate groups. The difference lies in n=3 for trimerization and n=2 for tetramerization. For a central chromium metal with a coordination number of six, the optimal structure of the catalytically active species for selective trimerization of ethylene is composed of three ethylene molecules and three dentate groups of the ligand coordinating to the central chromium simultaneously. In contrast, the catalytically active species for selective tetramerization of ethylene is composed of four ethylene molecules coordinating to chromium and the bidentate group of the ligand, which is consistent with the first insight.

A corollary derived from this is that the four π-ethylene molecules in the tetramerization active species can form two coordination configurations: first, the two dentate groups coordinate to two coordination sites perpendicular to each other, referred to as angular coordination; second, the two dentate groups coordinate to two coordination sites linearly aligned with the central metal, referred to as linear coordination.

The main structural difference between the catalytically active species of selective trimerization and selective tetramerization of ethylene lies in that the three π-ethylene molecules in the trimerization active species can form a planar transient conformation during molecular rotation, thereby forming a single catalytically active center and achieving high selectivity for the target product. In contrast, the four π-ethylene molecules in the active species of the selective tetramerization catalyst invented by Sasol in 2004 cannot form a planar transient conformation during molecular rotation, meaning they cannot form a single catalytically active center, resulting in diverse selectivities. Over the past two decades, all follow-up improvement studies have been limited to the scope of angular coordination configuration. Similarly, due to the inability of the four π-ethylene molecules in the angular coordination configuration to form a complete planar transient conformation, the results show that the selectivity of the target product 1-octene cannot exceed the ceiling of approximately 70% for any chromium-based catalyst with certain catalytic activity.

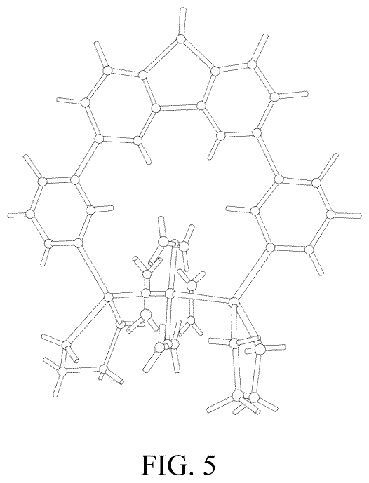

With this discovery, the idea of “constructing a tetramerizing catalytic active center where four π-ethylene molecules can form a complete planar transient conformation” was inevitably generated. Based on this idea, nearly 280 bulky and small-steric-hindrance bidentate and monodentate ligands were designed. The purpose of the bulky ligand design is to make the volume of the ligand in the catalytically active species sufficiently large, preventing its two dentate groups from coordinating to adjacent, perpendicular coordination sites of the chromium center and forcing or regulating them to coordinate to two coordination sites linearly aligned with the central chromium. Thus, the four π-ethylene molecules will naturally coordinate to the four coordination sites of the chromium center that can form a planar transient conformation, as shown in FIG. 5. Compared with Sasol's tetramerization catalytic model, this catalytic model is a completely new type, which will inevitably be reflected in the tetramerization catalytic performance.

In addition, the authorized Chinese invention patent “A Small Steric Hindrance Organophosphorus Ligand, Preparation Method and Use Thereof in Production of 1-Octene and 1-Hexene from Ethylene” (Patent No. 201710096548.4) invented by Li Dagang and Li Song has confirmed that reducing the steric hindrance in the microenvironment of the central metal chromium is beneficial to the formation of 1-octene in the products of ethylene selective oligomerization. The present disclosure continues this achievement and extends it to the technology of regulating the spatial geometric configuration of catalytically active species using bulky ligands, forming a completely new ethylene selective oligomerization catalytic model (FIG. 5). Innovations in the catalytic model will inevitably lead to changes in reaction results.

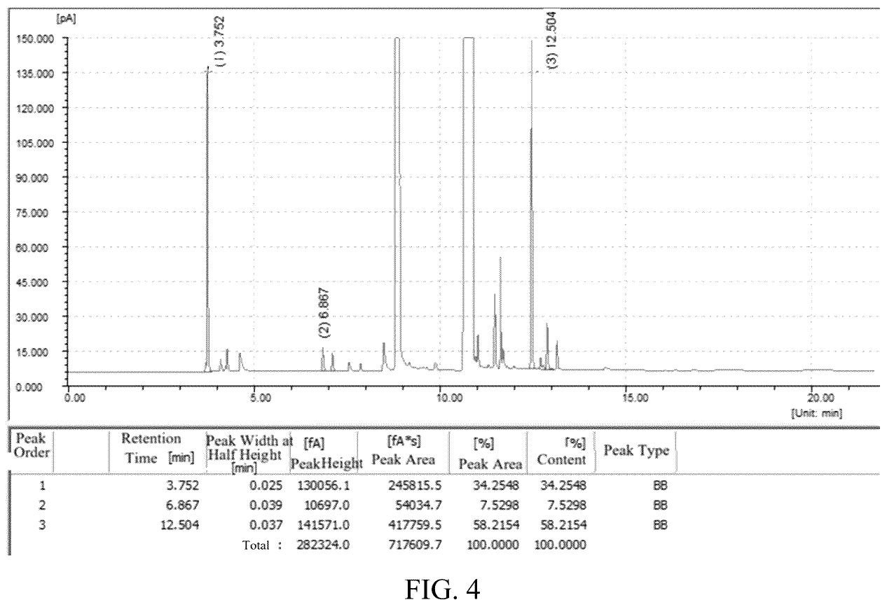

Experiments including catalyst synthesis, characterization, and tetramerization reaction evaluation show that the ethylene selective oligomerization catalytic system of this novel catalytic model has three new characteristics:
1. Ethylene selective oligomerization only generates three useful comonomers: 1-octene as the main product, and 1-hexene and 1-butene as by-products, with adjustable distribution.
2. The linear n-terminal olefin contents of the three comonomers are 99% for butene, 98% for hexene, and 97% for octene, respectively.
3. No waste higher olefins such as C10-C30 are produced, with carbon utilization rate approaching 100%. As shown in FIG. 4, the chromatogram contains four major peaks: the largest flat peak with a retention time of 10.70-11.00 is the peak of the solvent methylcyclohexane, and the other three peaks from left to right are the chromatographic peaks of 1-butene, 1-hexene, and 1-octene, respectively. There are no peaks corresponding to C10-C20 olefins.

The effects of these three characteristics are as follows: For ten-million-ton-scale polyolefin production enterprises, the wide range of polyolefin product grades requires various comonomers. Purchasing comonomers externally at high costs reduces the competitiveness of the enterprise's own brands. With the application of the present disclosure, if the enterprise produces comonomers in-house, it only needs to construct one set of oligomerization equipment using this catalyst or replace the catalyst of existing ethylene selective oligomerization equipment for single products (including dimerization, trimerization, or tetramerization equipment) with the catalyst of the present disclosure. By adjusting the distribution ratio of the three comonomers according to the technology disclosed in this patent, the demand for three different comonomers can be fully met. There is no need to separately construct or operate three sets of oligomerization equipment: ethylene tetramerization for octene production, ethylene trimerization for hexene production, and ethylene dimerization for butene production. Therefore, it can be called the “one-for-three” ethylene oligomerization catalytic technology, with obvious advantages.

The bidentate ligand composed of two identical or different dentate groups containing one or more dentate elements selected from P, N, S, and O linked by a bridging group that does not change configuration due to σ-rotation is a bidentate arch-shaped ligand connected by a double bond conjugated system. The bridging group linking the two identical or different dentate groups is a group with a sufficiently long double bond conjugated system.

The present disclosure first conducts basic theoretical research on in-situ high-pressure infrared spectroscopy of ethylene oligomerization reactions, captures structural information of the catalytically active species for ethylene trimerization, and based on this, combines multiple basic experimental studies by the inventors to deduce the catalytically active species for ethylene trimerization to 1-hexene. Experiments have confirmed the existence of this catalytically active species, which has been used to guide the successful innovation of catalysts. Since it is universally recognized that “selective tetramerization of ethylene and selective trimerization of ethylene follow the same reaction mechanism”, such catalytically active species are used to study ethylene tetramerization, revealing a series of inherent regular insights into selective tetramerization of ethylene reactions. These regular insights are used to guide the rational design and implementation of catalysts for selective tetramerization of ethylene to 1-octene, achieving the expected results.

### (I) Discovery of Catalytically Active Species for Ethylene Oligomerization

The in-situ infrared spectrum of the quaternary catalyst system composed of chromium(III) 2-ethylhexanoate (Cr(EH)₃), 2,5-dimethylpyrrole (DMP), triethylaluminum (TEA), and hexachloroethane (HCE) dissolved in n-heptane solvent under high-purity nitrogen protection shows that the main absorption peak is the absorption bands of n-heptane. Although the concentration of the catalyst components is very low, several weak peaks are observed: 1007 cm⁻¹ (Cr—N bond), 1082 cm⁻¹ (Al—N bond), and 1195 cm⁻¹ (Al—O or C—O bond), indicating that reactions have occurred between the four catalyst components.

After introducing 2.0 MPa of reactant ethylene for 3 minutes, nine new IR absorption peaks are observed in the spectrum:
Peak 1 (1641 cm⁻¹): Corresponding to the C═C stretching vibration of the reaction product 1-hexene.
Peak 2 (1523 cm⁻¹): Corresponding to Cr/π-ethylene of ethylene, i.e., the stretching vibration of ethylene coordinated to the central metal chromium.
Peak 3 (1466-1455 cm⁻¹): Corresponding to the antisymmetric bending vibration of 1-hexene (reaction product) and n-heptane (solvent).
Peak 4 (1380 cm⁻¹): Corresponding to the symmetric bending vibration of CH₃ in 1-hexene (reaction product) and n-heptane (solvent).
Peak 5 (1138 cm⁻¹): Unknown.
Peak 6 (1024 cm⁻¹): Corresponding to the Cr—N bond stretching vibration of DMP (catalyst) coordinated to Cr.
Peaks 7 and 8 (993 cm⁻¹ and 910 cm⁻¹): Both corresponding to the out-of-plane twisting IR absorption of ═CH₂ in the reaction product 1-hexene.
Peak 9 (773 cm⁻¹): Corresponding to the (CH₂)₄ skeleton vibration of the solvent n-heptane.

After 5 minutes of ethylene introduction, the test shows that due to the increase in reaction products, Peak 3 (1466-1455 cm⁻¹), corresponding to the antisymmetric bending vibration of 1-hexene and n-heptane, grows rapidly, turning the weak peak of π-ethylene (1523 cm⁻¹) into a shoulder peak.

The absorption bands of the ethylene oligomerization product 1-hexene and by-product PE (Peaks 1, 3, 4, 7, and 8) gradually increase, indicating that the ethylene oligomerization reaction is proceeding.

At the same time, it is shown that after introducing ethylene, Peak 6 (1007 cm⁻¹, Cr—N bond) shifts to 1024 cm⁻¹ (a shift of 17 cm⁻¹), and the Al—N bond peak (1082 cm⁻¹) shifts to 1069 cm⁻¹. Surprisingly, a shoulder peak is detected at 1523.55 cm⁻¹ one minute after introducing ethylene, which grows into an independent weak absorption peak after 3 minutes of reaction. When the reaction is extended to 5 minutes, the rapid growth of Peak 3 (1466-1455 cm⁻¹, corresponding to the antisymmetric bending vibration of 1-hexene (reaction product) and n-heptane (solvent)) turns the 1523.55 cm⁻¹ peak back into a shoulder peak. Verification confirms that the 1523.55 cm⁻¹ peak should be assigned to the stretching vibration peak of π-ethylene formed after ethylene coordinates to the central metal chromium. This is the first global report of evidence for the existence of π-ethylene in ethylene oligomerization reactions. In-situ high-pressure infrared spectroscopy tests of ethylene trimerization show that during the ethylene trimerization reaction, 2,5-dimethylpyrrole and π-ethylene dissolved in n-heptane coordinate simultaneously to the central chromium metal with a coordination number of six, forming a complex: (2.5-DMP)ₙCr(π-C₂H₄)₆₋ₙ. N/Cr ratio experiments show that the catalytic performance is optimal when N/Cr=3 (mol), and the complex can be written as (2.5-DMP)₃Cr(π-C₂H₄)₃.

The coordination of three 2.5-DMP molecules to the central metal Cr does not exclude the formation of the coordination configuration shown in FIG. 2. Calculations indicate that three π-ethylene molecules in angular coordination are more favorable for the homopolymerization to form 1-hexene than those in facial coordination. The reason why adding halides to the ternary catalyst system for ethylene trimerization significantly improves selectivity is that halides promote the dominance of the angular coordination configuration.

Based on the tested complex (2.5-DMP)₃Cr(π-C₂H₄)₃ as the catalytically active species, combined with experimental results from the inventors' studies on the role of halides, by-product formation mechanism, tracer atom experiment calculations, and catalyst deactivation mechanism investigation, the catalytically active species for selective trimerization of ethylene to 1-hexene is proposed.

In the prior art, the chromium-based ternary catalyst composed of chromium(III) 2-ethylhexanoate/2,5-dimethylpyrrole/triethylaluminum has an ethylene trimerization selectivity to 1-hexene of only 34.7%. Adding alkyl halides as the fourth catalyst component can increase the selectivity of 1-hexene to 92-98%. W. K. Reagan (Phillips Petroleum Company, EP 0608447, 1994) defined the added alkyl halides as those containing three or more halogen elements on one carbon atom; Yoshitake Araki, E. Tanaka, H. Urata (Mitsubishi Chem. Co, U.S. Pat. No. 5,750,817, 1998) defined them as those containing three or more halogen elements on two adjacent carbon atoms. Using (2.5-DMP)₃Cr(π-C₂H₄)₃ as the catalytically active species, the inventors of the present patent revealed that polyhalogenated compounds with a tetrahedral configuration act as adjustable site-occupying agents, regulating the formation of the dominant angular coordination configuration of three ethylene molecules, thereby significantly improving the selectivity of 1-hexene, which is called the site-occupying effect. Investigation into the causes of catalyst deactivation confirms this inference. After 40 minutes of reaction, the catalyst is deactivated, and the content of hexachloroethane in the reaction solution is significantly reduced to only about 20% of the initial amount. Meanwhile, it is found that most of the initial hexachloroethane is converted into tetrachloroethylene and a small amount of trichloroethylene during the reaction (as shown in the following formula):

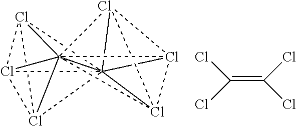

Hexachloroethane with Tetrahedral Structure Before Trimerization Reaction
Tetrachloroethylene with Planar Structure after Trimerization Reaction

It is well known that polyhalogenated alkanes can be converted into polyhalogenated alkenes via the following reaction formula: ClCl₃-CCl₃+(C₂H₅)₃Al [symbol] Cl₂C═CCl₂+(C₂H₅)₂AlCl+ClC₂H₅.

Applied Catalysis A: General 481 (2014) 39-48 reported the results of ethylene trimerization using tetrachloroethylene instead of hexachloroethane as the fourth catalyst component. The catalytic activity decreased to 38 kg/gCr·h, and the selectivity to 1-hexene was only 66%. Although tetrachloroethylene contains three or more halogen elements on two adjacent carbon atoms, consistent with the definition of Yoshitaka Araki of Mitsubishi Corporation, its planar structure cannot exert the site-occupying effect for angular coordination on the central metal Cr, leading to catalyst deactivation. This confirms that only polyhalogenated compounds with a tetrahedral structure as the fourth component of the quaternary chromium-based catalytic system can significantly improve the selectivity and catalytic activity of selective trimerization of ethylene to 1-hexene.

The Phillips chromium-based catalyst for ethylene trimerization to 1-hexene meets industrial requirements in terms of selectivity and catalytic activity but has a short catalytic activity period (only about 30 minutes), resulting in high consumption of one-time use catalysts. Extending the catalyst activity period and reducing catalyst consumption with newly discovered “catalytically active species” are important challenges. The Chinese patent CN1256968A (1999) invented by Li Dagang and Du Xiangdong won the Excellent Award of the 19th China Patent Award. The innovative idea is to find polyhalogenated compounds with better stability than the aliphatic polyhalogenated alkanes used in the prior art as site-occupying agents. Taking this as a breakthrough, the dynamic concept of single bond rotation in molecules is introduced, and a series of aromatic polyhalogenated compounds with good stability are designed as site-occupying agents. For example, 2-fluoro-6-chlorobenzotrichloride (FCTCT) can extend the activity period from 30 minutes to more than 180 minutes while maintaining high selectivity and activity. It won the China Patent Excellent Award (Patent No. ZL99120585.5, 2017), supporting the industrialization of selective trimerization of ethylene to 1-hexene in China at the basic theoretical and technical levels.

The successful innovation of the selective hexene trimerization catalyst composed of stable aromatic polyhalogenated compounds as site-occupying agents and the acquisition of the China Patent Excellent Award (No. ZL99120585.5, 2017) fully confirm the accuracy of the hypothesis for selective trimerization of ethylene that “trimerization requires three ethylene molecules to coordinate to the central metal chromium simultaneously” and the corresponding “catalytically active species”.

It is well known to those skilled in the art that selective tetramerization of ethylene to 1-octene and selective trimerization of ethylene to 1-hexene share the same reaction mechanism. Therefore, complexes of central metal chromium and ethylene with similar structures can be used as catalytically active species, with only the coordination number of ethylene changed from three to four. Thus, the molecular formula of the catalytically active species for selective tetramerization of ethylene should be L₂Cr(π-C₂H₄)₄ (where L₂ is a bidentate ligand).

Using the site-occupying effect to study the structure-activity relationship of tetramerization catalytically active species, new theoretical insights are gained: Selective tetramerization of ethylene to 1-octene requires four π-ethylene molecules to coordinate to the central metal chromium simultaneously. For a central chromium metal with a coordination number of six (octahedral configuration), two coordination sites are occupied by the bidentate group of the ligand, and the other four coordination sites are coordinated by four π-ethylene molecules. In contrast, selective trimerization of ethylene requires three coordinated π-ethylene molecules, which can form a stable planar conformation, an essential condition for achieving high activity and selectivity in selective trimerization of ethylene.

For selective tetramerization of ethylene, both monodentate and bidentate ligands can form two types of coordination with the central chromium metal (coordination number six):

(1) The bidentate group of the ligand undergoes angular coordination at approximately 90°; (2) The bidentate group of the ligand undergoes linear coordination at 180°.

To date, the PNP-type and PRP-type bidentate ligands invented by Sasol and various modified bidentate ligands all coordinate to two perpendicular coordination sites. This coordination structure makes it difficult for the four π-ethylene molecules to form a coplanar conformation, resulting in low selectivity for the target product 1-octene in selective tetramerization of ethylene.

The inventors Li Dagang and Li Song disclosed a small steric hindrance organophosphorus ligand for selective tetramerization of ethylene to 1-octene in the Chinese patent application CN201710096548.4 (Small Steric Hindrance Organophosphorus Ligand and Preparation Method and Use Thereof in Production of 1-Octene and 1-Hexene from Ethylene, Publication No.: CN108456228A, Aug. 28, 2018), which achieved good results. Based on this invention, the present patent further innovates by adding the concept of regulating the spatial geometric configuration of the catalytically active species for selective tetramerization of ethylene. It modulates the bidentate group to coordinate to two coordination sites linearly aligned with the central metal, enabling four ethylene molecules to coordinate to the four coordination sites of the chromium center that can form a planar transient conformation, thereby constructing a novel ethylene selective oligomerization catalytic model, as shown in FIG. 5.

The technical measures of the present application are as follows: monodentate ligands with sufficiently large spatial volume or bidentate ligands with sufficiently long linking chains are designed, making it difficult for the two monodentate ligands or the bidentate group of the bidentate ligand to undergo angular coordination. Instead, they tend to self-coordinate to the coordination sites at both ends linearly aligned with the central chromium metal (i.e., linear coordination), facilitating the formation of a planar transient conformation by the four π-ethylene molecules. Furthermore, sufficiently small dentate groups and their substituents are designed to reduce rigidity and instantaneous interference with the self-assembly of π-ethylene into ∝-olefins, maintaining the state of a single catalytically active center as much as possible. Based on this unique idea and two novel measures, the innovative thinking and theoretical basis of the present disclosure are established, and a novel ethylene tetramerization catalytic model is constructed, as shown in FIG. 5.

The bulky and small-steric-hindrance ligand in the present application is a monodentate bulky and small-steric-hindrance ligand constructed from dentate groups consisting of dentate elements without substituents, a monodentate bulky and small-steric-hindrance ligand constructed from dentate groups consisting of dentate elements containing branched substituents, a bidentate ligand composed of two dentate groups of P or N linked by a bridging group, a bidentate ligand composed of two identical or different dentate groups containing one or more dentate elements selected from P, N, S, and O linked by a bridging group that does not change configuration due to σ-rotation, the bridging group has a double-bonded conjugated system; the dentate elements are one or more of P, N, S, and O; the monodentate bulky and small-steric-hindrance ligand constructed from dentate groups consisting of dentate elements containing branched substituents is specifically one or more of the following: a monodentate tertiary phosphine ligand compound containing only one dentate group, a monodentate tertiary amine ligand compound containing only one dentate group, a monodentate thioheterocyclic ligand compound containing only one dentate group, a monodentate oxoheterocyclic ligand compounds containing only one dentate group; the bidentate ligand composed of two identical or different dentate groups containing one or more dentate elements selected from P, N, S, and O linked by a bridging group that does not change configuration due to σ-rotation is a bidentate ligand compound composed of two dentate groups linked by a bridging group, wherein the two dentate groups of the bidentate ligand are located at two coordination sites that can form a straight line with the central metal, and the ligand and the central metal form an arch-shaped structure, providing sufficient operational space for the self-assembly of the four π-ethylene groups located at the central metal into the target product 1-octene.

The monodentate bulky and small-steric-hindrance ligand constructed from dentate groups consisting of dentate elements without substituents is as follows:

| Serial No. | | Skeletal | Substituents and |
|---|---|---|---|
| (Example) | Ligand | Formula | Coefficients |
| 1 (1) | 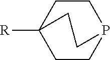 | C₇H₁₂P | R = Cph₃, CEt₃, (CH₂)nCH₃, Ph(CH₂)nCH₃, C₆H₁₁(CH₂)nCH₃, PhCOOR |
| 2 (1) | 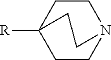 | C₇H₁₂N | R = Cph₃, CEt₃, (CH₂)nCH₃, Ph(CH₂)nCH₃, C₆H₁₁(CH₂)nCH₃ |
| 3 (4) | 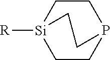 | C₆H₁₂PSi | R = Cph₃, (CH₂)nCH₃, CEt₃, Ph(CH₂)nCH₃ |
| 4 (5) | 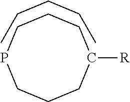 | C₁₀H₁₈P | R = Cph₃, (CH₂)nCH₃, CEt₃, Ph(CH₂)nCH₃ |
| 5 (6) | 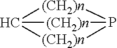 | — | n = 3, 4, 5, 6, 7 |
| 6 (6) | 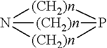 | — | n = 3, 4, 5, 6, 7 |
| 7 (6) | 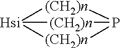 | — | n = 3, 4, 5, 6, 7 |
| 8 (7) | 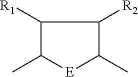 | C₁₀H₁₈ | R₁ = R₂ = Et, Pr E = S, O |
| 9 (7) | 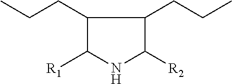 | C₁₀H₁₉N | R₁ = R₂ = H, Me |
| 10 (8) | 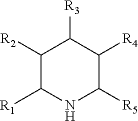 | C₅H₅S | R₁ = R₅ = H, Me R₂ = R₄ = H, Et, Pr, t-Bu R₃ = H, Pr, Cph₃, AlEt₂ |
| 11 (8) | 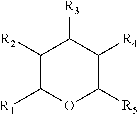 | C₅H₅O | R₁ = R₅ = H, Me R₂ = R₄ = H, Et, Pr, t-Bu R₃ = H, Pr, t-Bu, Cph₃ |
| 12 (8) | 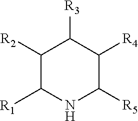 | C₅H₆N | R₁ = R₅ = H, Me R₂ = R₄ = H, Et, Pr, t-Bu R₃ = H, Pr, t-Bu, Cph₃ |
| 13 (9) | 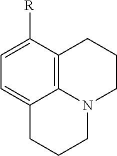 | C₁₂H₁₄N | R = Cph₃, CEt₃, Cpr₃, CMe₃ |
| 14 (10) | 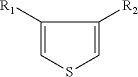 | C₄H₂S | R₁ = R₂ = Cph₃, CMe₃, CEt₃, Cpr₃, AlEt₂, Al(iBu)₂ |
| 15 | 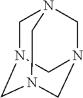 | C₆H₁₂N₄ | — |
| 16 | 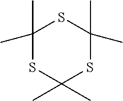 | C₉H₁₈S₃ | — |
| 17 (11) | 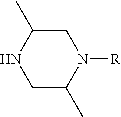 | C₆H₁₃N₂ | R = cph₃, CEt₃, AlEt₂, Al(iBu)₂, C(t-Bu)₃ |
| 18 (110 | 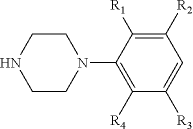 | C₁₁H₁₂N₂ | R₁ = R₄ = H, Me R₃ = R₂ = Pr t-Bu |
| 19 | 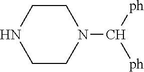 | C₁₇H₂₀N₂ | — |
| 20 | 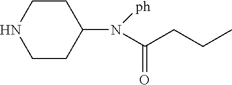 | C₁₄H₂₀N₂O | — |
| 21 (12) | 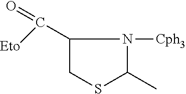 | C₅H₇O₂NS | — |
| 22 (12) | 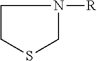 | C₃H₆NS | R = AlEt₂, Ali-Bu₃, cph₃, cpr₃ |
| 23 (12) | 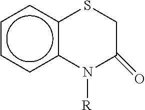 | C₈H₆NOS | R = cph₃, CEt₃, cpr₃, C(t-Bu)₃ |
| 24 (120 | 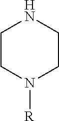 | C₅H₁₁N₂ | R = cph₃, CEt₃, AlEt₂, Al(i-Bu)₂ |
| 25 | 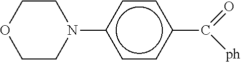 | C₁₇H₁₇NO₂ | — |
| 26 (13) | 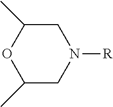 | C₆H₁₂NO | R = cph₃, CEt₃, Cpr₃, C(t-Bu)₃, Al(Bu)₃ |
| 27 (13) | 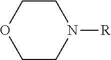 | C₄H₈NO | R = cph₃, CEt₃, Cpr₃, C(t-Bu)₃, Al(Bu)₃ |
| 28 (13) | 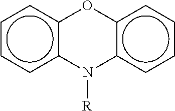 | C₁₂H₈NO | R = cph₃, CEt₃, AlEt₂, Cpr₃, C(t-Bu)₃, Al(i-Bu)₂ |
| 29 (13) | 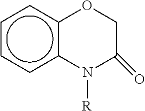 | C₈H₆NO₂ | R = cph₃, CEt₃, AlEt₂, Cpr₃, C(t-Bu)₃, Al(i-Bu)₂ |
| 30 (13) | 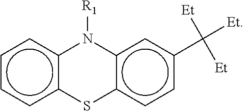 | C₁₉H₂₂NS | R = cph₃, AlEt₂, CEt₃ |

The monodentate bulky and small-steric-hindrance ligand constructed from dentate groups consisting of dentate elements containing branched substituent is as follows:

| | |
|---|---|
| Serial No. | Skeletal |

| (Example) | Ligand | Formula | Substituents and Coefficients | | | |
|---|---|---|---|---|---|---|
| 31 (14) | 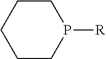 | PC₅H₁₀ | 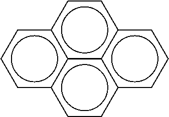 | 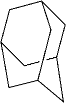 | 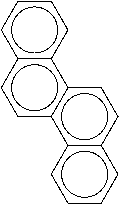 | 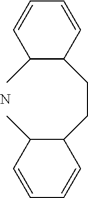 |
| 32 (14) | 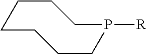 | PC₇H₁₄ | 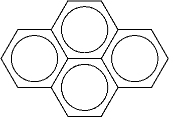 | 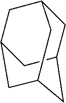 | 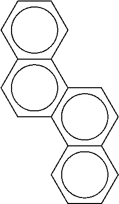 | 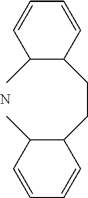 |
| 33 (14) | 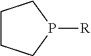 | PC₄H₈ | 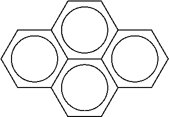 | 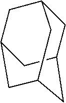 | 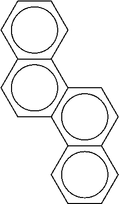 | 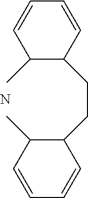 |
| 34 (15) | 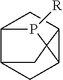 | PC₇H₁₀ | 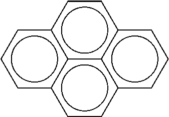 | 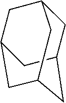 | 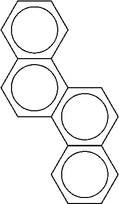 | 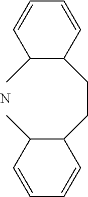 |
| 35 | 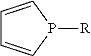 | PC₄H₄ | 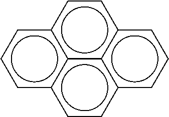 | 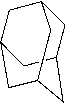 | 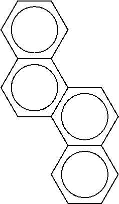 | 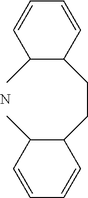 |
| 36 (16) | 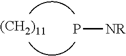 | PC₁₁H₂₂ | NR = 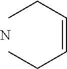 | 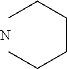 | 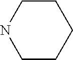 | 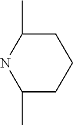 |

| | | |
|---|---|---|
| 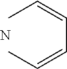 | 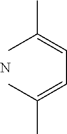 | 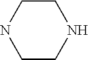 |

| | | | |
|---|---|---|---|
| 37 (17) | 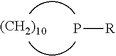 | PC₁₀H₂₀ | R = Me, Et, Pr, Ph, C₄H₁₁ = 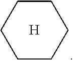 |

The bidentate ligand composed of two dentate groups of P or N linked by a bridging group is:

| Seri - | | | | | |
|---|---|---|---|---|---|
| al | | | | | |
| No. | | | | | |
| (Ex- | | | | | |
| am- | | | Dental Group and | | |
| ple) | Ligand | Skeletal Formula | Coefficient | | |
| 38 (18) | CP₁—(CH₂)n—CP₂ | —(CH₂)n— | cp₁ = 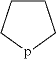 | 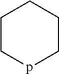 | pph₂ |

| | | | cp₂ = 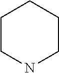 | |
|---|---|---|---|---|
| | | | 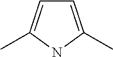 | |
| | | | n = 3, 5, 7, 9, 4 | |
| | | | cp₁ = cp₂ or cp₁ ≠ cp₂ | |
| 39 (19) | CP—N(Me)—(CH₂)ₙ—N(Me)—CP | —(Me)N—(CH₂)ₙN(Me)— | CP = 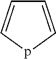 | 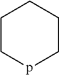 |
| | | | 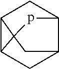 | pph₂ 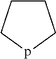 |
| | | | C₇H₁₀P | |
| | | | n = 3, 4, 5, 6, 7 | |
| 40 (19) | CPNH(CH₂)O(CH₂)O(CH₂)NHCP | C₈H₁₈N₂O₂ | CP = 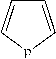 | 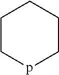 |

| 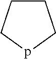 | |
|---|---|
| 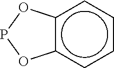 | |
| 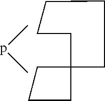 | pph₂ |

| | | | | |
|---|---|---|---|---|
| 41 (19) | 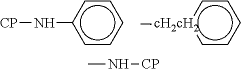 | C₁₄H₁₄N₂ | CP = 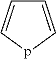 | 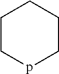 |

| | |
|---|---|
| 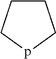 | 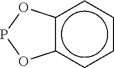 |

| | | | 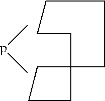 | |
|---|---|---|---|---|
| | | | pph2 | |
| 42 (19) | CP—NH—(CH₂)₃—NH—(CH₂)₃—NH—CP | C₆H₁₅N₃ | CP = 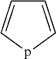 | 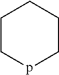 |

| | |
|---|---|
| 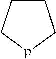 | 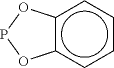 |

| | |
|---|---|
| 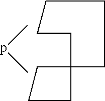 | pph₂. |

The bidentate ligand composed of two identical or different dentate groups containing one or more dentate elements selected from P, N, S, and O linked by a bridging group that does not change configuration due to σ-rotation is:

| Seri - | | | | | |
|---|---|---|---|---|---|
| al | | | | | |
| No. | | | | | |
| (Ex- | | | | | |
| am- | | Skeletal | | | |
| ple) | Ligand | Formula | Dental Group and Coefficient | | |
| 43 (20) | 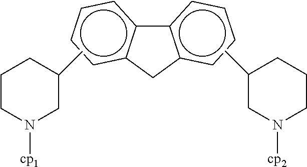 | C₂₃H₂₆N₂ | cp₁ = cp₂ 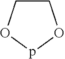 pph₂ | 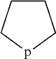 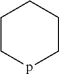 | 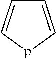 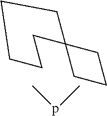 |
| 44 (20) | 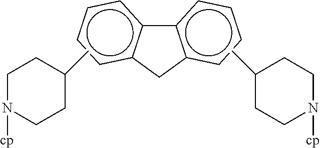 | C₂₃H₂₆N₂ | cp₁ = cp₂ 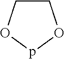 pph₂ | 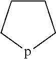 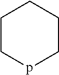 | 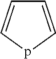 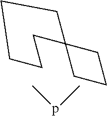 |
| 45 (21) | 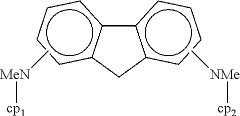 | C₁₅H₁₄N₂ | cp₁ = cp₂ 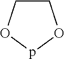 pph₂ | 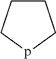 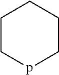 | 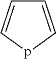 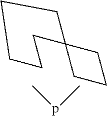 |
| 46 (22) | 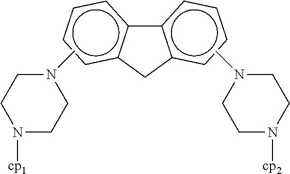 | C₂₁H₂₄N₄ | cp₁ = cp₂ 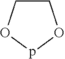 pph₂ | 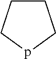 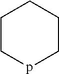 | 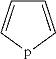 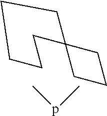 |
| 47 (23) | 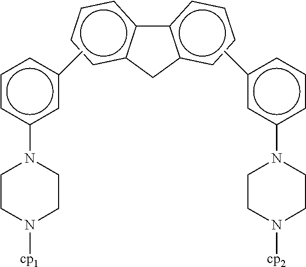 | C₃₃H₃₂N₄ | cp₁ = cp₂ = 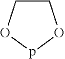 pph₂ | 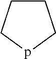 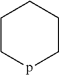 | 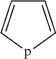 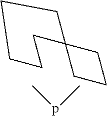 |
| 48 (24) | 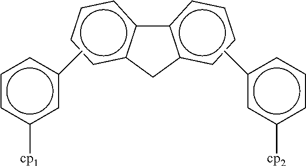 | C₂₅H₁₅ | cp [symbol] ≠ cp₂ cp₁ = cp₁ = 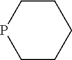 | 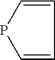 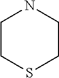 | 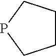 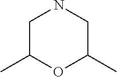 |
| | | | 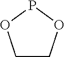 | pph₂ | 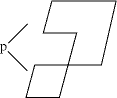 |
| | | | 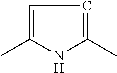 | 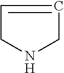 | 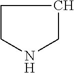 |
| | | | 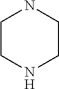 | | |
| 49 (24) | 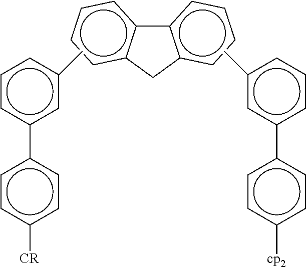 | C₃₇H₂₄ | cp [symbol] ≠ cp [symbol] cp [symbol] = cp [symbol] = 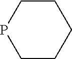 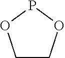 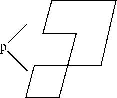 | 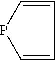 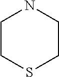 pph₂ 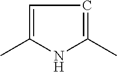 | 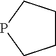 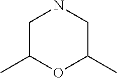 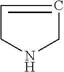 |

| | | | 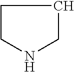 | | 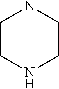 |
|---|---|---|---|---|---|
| 50 (25) | 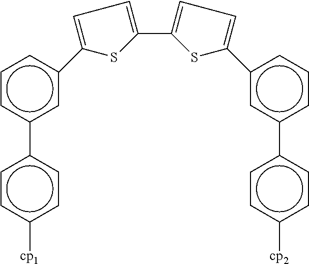 | C₃₂H₂₀S | cp [symbol] = cp [symbol] = cp [symbol] = cp₂ = 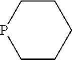 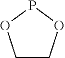 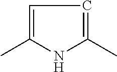 | 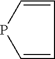 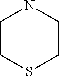 pph₂ 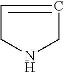 | 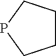 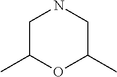 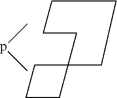 |

| | | | 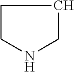 | 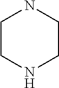 | |
|---|---|---|---|---|---|
| 51 (26) | 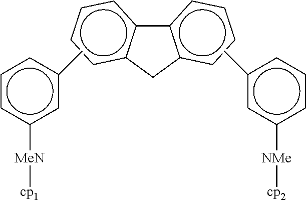 | C₂₇H₂₂N₂ | 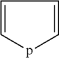 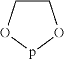 | 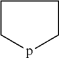 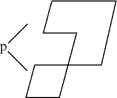 | 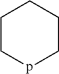 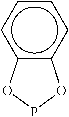 |
| | | | | pph₂ | |
| 52 (26) | 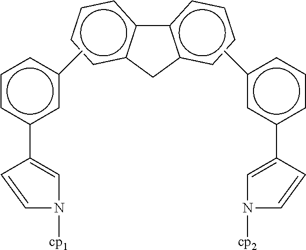 | C₃₃H₁₃N₂ | cp₁ = cp₂ 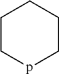 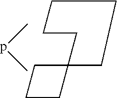 | 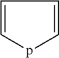 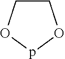 pph₂ 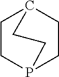 | 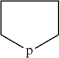 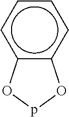 |
| 53 (27) | 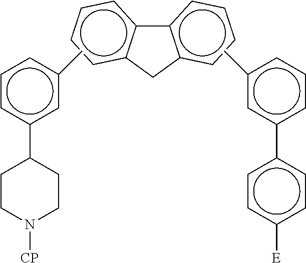 | C₃₁H₂₉N | cp = 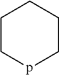 | 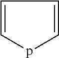 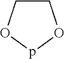 | pph₂ |
| | | E = | 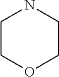 | 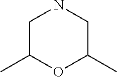 | 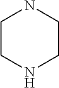 |
| | | | 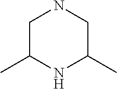 | 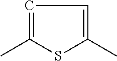 | 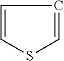 |

| | | | | |
|---|---|---|---|---|
| | | | 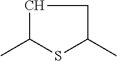 | 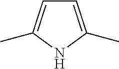 |
| 54 (28) | 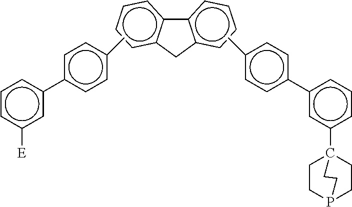 | C₄₄H₃₆P | E = 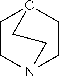 | 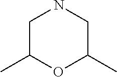 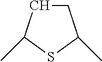 |

| | | | | | |
|---|---|---|---|---|---|
| 55 (29) | 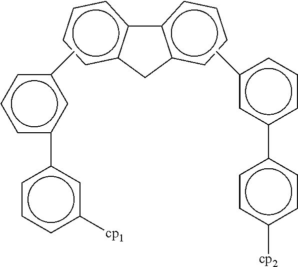 | C₃₇H₂₄ | cp₁ = cp₂ = 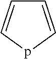 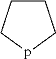 | 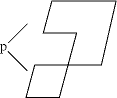 pph₂ | 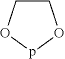 |
| 56 (29) | 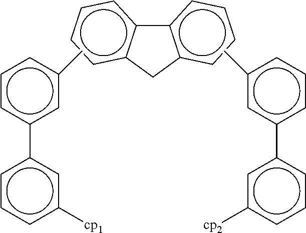 | C₃₇H₂₄ | cp₁ = cp₂ = 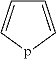 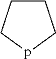 | 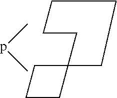 pph₂ | 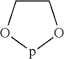 |

| | | | | |
|---|---|---|---|---|
| 57 (30) | 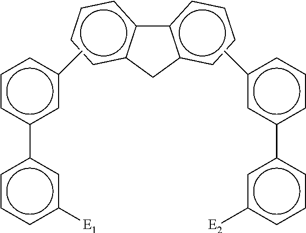 | C₃₇H₂₄ | E₁ = E₂ or 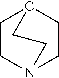 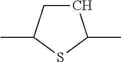 | E₁ ≠ E₂ 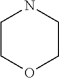 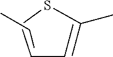 |

| | | | |
|---|---|---|---|
| 58 (31) | 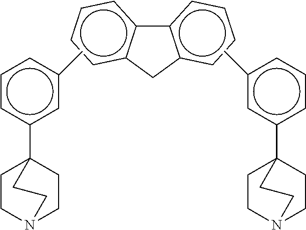 | C₂₇H₃₃N₂ | — |
| 59 (31) | 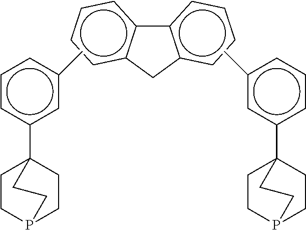 | C₂₇H₃₃P₂ | — |
| 60 (31) | 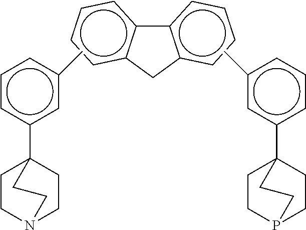 | C₂₇H₃₃NP | — |

[symbol]

indicates data missing or illegible when filed

The dentate groups are specifically as follows:
Dentate group 1, molecular formula: PPh₂;
Dentate group 2, molecular formula: C₈H₁₄P, structural formula:

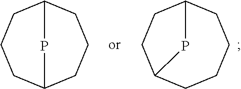

Dentate group 3, molecular formula: PC₄H₈;
Dentate group 4, molecular formula: PC₅H₁₀, structural formula:

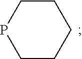

Dentate group 5, molecular formula: PC₆H₁₀O₂, structural formula:

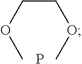

Dentate group 6, molecular formula: PC₇H₁₂, structural formula:

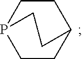

Dentate group 7, molecular formula: NC₇H₁₂, structural formula:

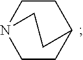

Dentate group 8, molecular formula: NC₄H₈S, structural formula:

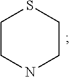

Dentate group 9, molecular formula: NC₄H₄;
Dentate group 10, molecular formula: NC₄H₆;
Dentate group 11, molecular formula: N₂C₄H₉, structural formula:

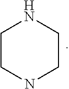

Use of the bulky and small-steric-hindrance ligand in a ternary catalytic system, wherein the ternary catalytic system is a ligand/transition metal/activator ternary catalytic system composed of a bulky and small-steric-hindrance ligand, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

The activator is an aluminum-containing activator, and the ternary catalytic system catalyzes the selective oligomerization of ethylene to produce three linear normal α-olefin products, generating only three linear normal α-olefin products of 1-octene, 1-hexene, and 1-butene, without generating C10-C20 waste olefins, and the arbon utilization rate is 100%.

The aluminum-containing activator is an alkylaluminum compound and/or an alkoxyaluminum compound, the alkylaluminum compound is specifically one or more of triethylaluminum, trimethylaluminum, and triisobutylaluminum, the alkoxyaluminum compound is specifically one or more of MAO, MMAO, DMAO, and MMAO-3A.

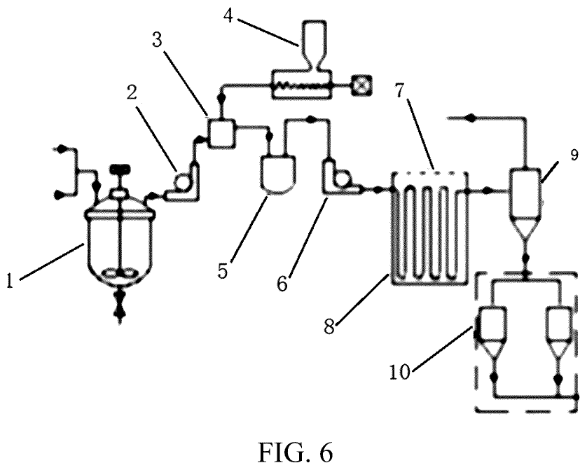

The technological process of the industrial manufacturing method for phosphabicyclic secondary phosphine compounds in in bulky and small-steric hindrance ligands is shown in FIG. 6:

Degassed norbornadiene or 1,5-cyclooctadiene is injected into premixer 1 at a flow rate of 0.6 kg/h, mixed with degassed solvent at a flow rate of 1.4 kg/h. The mixture is then pumped into gas-liquid intensive mixer 3 by a metering pump 2 at a flow rate of 2.0 kg/h under low temperature (5° C.). Meanwhile, azobisisobutyronitrile (AIBN) initiator is added via an oxygen-free initiator feeder 4, and the system is maintained at a temperature of 2-8° C. Phosphine (PH₃) gas is injected into the mixer, with the pressure kept at 1.6 MPa. The gas-liquid intensive mixer is activated to enable saturated absorption of PH₃ by the liquid phase. The PH₃-saturated absorption solution is stored in a saturated liquid storage tank 5 under conditions of ≤2-8° C. and 1.6 MPa. Using a diaphragm pump 6, the saturated absorption mixture is injected into a pipeline reactor 7 at a flow rate of 2.01 kg/h. A water bath device 8 is used to maintain the temperature inside the reactor at 70-80° C. and pressure at 3.0 MPa. The effluent from the pipeline reactor flows into a gas-liquid separator 9, where a trace amount of unreacted PH₃ gas is separated, recycled, and stored in a liquid nitrogen cryogenic storage and purification device. The conversion rate of 1,5-cyclooctadiene reaches 92%; the capillary gas chromatography analysis shows the concentration of polycyclic phospholane formed in the reaction solution is 25-30% (by weight), with a selectivity of >90%. The liquid phase is stored in the intermediate storage tank 10 for subsequent use. This process has the following advantages:
1) Gaseous PH₃ can be converted to liquid under low pressure, enabling accurate metering.
2) It avoids potential safety hazards caused by excessive toxic gaseous PH₃ in the reaction system.
3) The use of a pipeline reactor for highly toxic substances ensures high safety and reliability.

### Example 1

A bulky and small-steric-hindrance ligand, whether a monodentate ligand or a bidentate ligand with small steric hindrance, must occupy a sufficiently large spatial space. This structural feature prevents the two dentate groups from coordinating to two adjacent coordination sites of the central metal; instead, the dentate groups have to coordinate to two diagonal coordination sites, thereby forming a bulky and small-steric-hindrance ligand compound.

A method for preparing the bulky and small-steric-hindrance ligand P(CH₂CH₂)₃C—R (where R=CPh₃, CEt₃, CPr₃, or C(C₆H₁₁)₃) adopted a lithiation method, also known as the Wurz method. The synthetic route when R=CPh₃ was as follows:

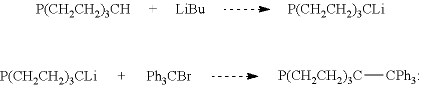

Synthesis of Ph₃C—(C₇H₁₂P):
Step 1: To a Schlenk flask containing 3 mL of THF solution of P(CH₂CH₂)₃CH, a 1.6 M hexane solution of BuLi (4.5 mL, 7.2 mmol) was added dropwise within 5 minutes. The mixture was stirred at −78° C. for 40 minutes while maintaining the temperature.
Step 2: 2 mL of THF solution containing 2.6 mmol of triphenylmethyl bromide (Ph₃CBr) was added to the flask.
Step 3: The mixture was warmed to room temperature overnight. The reaction mixture was extracted with 40 mL of anhydrous diethyl ether, and the organic phase was dried over sodium carbonate. P(CH₂CH₂)₃C—CPh₃ was obtained via vacuum distillation with a yield of 50%.

Compounds (PC₇H₁₂)CEt₃, (PC₇H₁₂)CPr₃, and (PC₇H₁₂)C(C₆H₁₁)₃ could be prepared by substituting Ph₃CBr with BrCEt₃, BrCPr₃, or BrC(C₆H₁₁)₃, respectively.

Method for preparing N(CH₂CH₂)₃C—R: the target product was synthesized by substituting P(CH₂CH₂)₃CH with N(CH₂CH₂)₃CH in the above procedure.

The performance evaluation of the ligand prepared in this example in ethylene-to-target-product molecular self-assembly featuring low steric hindrance and minimal interference was detailed in Example 32.

The dentate groups possessed small-steric-hindrance heterocyclic or aryl structures that minimally interfered with π-ethylene self-assembly. The linear n-terminal olefin ratios of the three alkene products were as follows: 1-butene: 99% (by weight); 1-hexene: 98% (by weight); 1-octene: 97% (by weight).

No byproducts such as alkenes or alkanes with 10 or more carbon atoms were detected, and the effective carbon utilization rate was nearly 100%.

### Example 2

A laboratory method for preparing small-steric-hindrance phosphacyclic compounds was provided via free radical addition of non-conjugated alkenes. The small-steric-hindrance phosphacyclic compounds were secondary phosphines of phosphacycloalkanes (HPR), including H(C₅H₁₀P) (6-phosphacyclohexane), H(C₇H₁₀P) (8-phosphatricyclooctane), H(C₈H₁₄P) (9-phosphabicyclononane), H(C₈H₁₆P) (9-phosphamonocyclononane), H(C₉H₁₉P) (10-phosphamonocyclodecane), H(C₁₀H₂₀P) (11-phosphamonocycloundecane), 1-phosphabicyclo[2.2.2]heptane, {HC[(CH₂)ₙ]₃P}, and 8-phosphatricyclooctane. The laboratory method for preparing H(C₅H₁₀P) (6-phosphacyclohexane) included the following steps:
Step 1: Under the protection of high-purity nitrogen, 6.8 g (0.1 mol) of 1,4-pentadiene was weighed and dissolved in 100 mL of toluene. The solution was transferred to a 200 mL autoclave equipped with magnetic stirring;
Step 2: 0.01 mol of azobisisobutyronitrile (AIBN) initiator was added to the autoclave. PH₃ gas was charged to a pressure of 3.0 MPa, and stirring was initiated. The reaction temperature was controlled at 70-80° C. After the reaction pressure ceased to decrease, the reaction was continued for an additional 30 minutes before termination;
Step 3: After recovering the PH₃ gas, high-purity nitrogen was charged for subsequent use. The liquid sample was analyzed by chromatography to determine the content of the cyclic secondary phosphine compound H(PC₅H₁₀). The yield of the target product H(PC₅H₁₀) was calculated as 90% (based on 1,4-pentadiene), with a ³¹P NMR chemical shift of −65 ppm.

### Example 3

A method for industrially manufacturing heterocyclic secondary phosphine derivatives in a bulky and small-steric-hindrance ligand was provided. The heterocyclic secondary phosphine derivatives included 6-phosphacyclohexane, 9-phosphamonocyclononane, 10-phosphamonocyclodecane, 11-phosphamonocycloundecane, 4-phosphabicyclo[2.2.2]triethylmethylphosphine, 5-phosphabicyclo[3.3.3]tripropylmethylphosphine, 6-phosphabicyclo[4.4.4]tributylmethylphosphine, 7-phosphabicyclo[5.5.5]tripentylmethylphosphine, and 9-phosphabicyclononylphosphine. The method for industrially manufacturing 9-phosphabicyclononylphosphine included the following steps:
Step 1: Degassed norbornadiene or 1,5-cyclooctadiene was injected into the premixer 1 at a flow rate of 0.6 kg/h, mixed with degassed solvent at 1.4 kg/h, and then pumped into the gas-liquid intensive mixer 3 at 2.0 kg/h via the metering pump 2 at 5° C. Simultaneously, AIBN initiator was added using the oxygen-free initiator feeder 4, and the temperature was maintained below 5° C. PH₃ gas was injected, and the pressure was kept at 1.6 MPa.
Step 2: The gas-liquid intensive mixer was activated to achieve saturated absorption of PH₃ by the liquid phase. The saturated absorption solution was stored in the saturated liquid storage tank 5 at ≤5° C. and 1.6 MPa. The saturated absorption mixture was pumped into the pipeline reactor 7 at 2.01 kg/h via the diaphragm pump 6. The temperature in the pipeline was maintained at 70-80° C. by the water bath device 8, and the pressure was controlled at 3.0 MPa.
Step 3: The mixture flowed from the outlet of the pipeline reactor into the gas-liquid separator 9, where a small amount of unreacted PH₃ gas was separated, recovered, and stored in a liquid nitrogen cryogenic storage and purification system. The conversion rate of 1,5-cyclooctadiene was 92%, and the concentration of the polycyclic phosphine alkane produced in the reaction liquid analyzed by capillary chromatography was 25-30% (by weight) with a selectivity of >90%. The liquid phase was stored in the intermediate storage tank 10 for subsequent use.

1,5-Pentadiene, 1,7-octadiene, 1,8-nonadiene, or 1,9-decadiene could be used as substitutes for 1,5-cyclooctadiene.

### Example 4

A method for preparing the bulky and small-steric-hindrance ligand R—(SiCH₂CH₂CH₂)₃N (where R=CPh₃, CEt₃, CPr₃, or C(C₆H₁₁)₃) adopted a lithiation method, also known as the Wurz method. The synthetic route in Example 1 was employed, P(CH₂CH₂)₃CH was substituted with H[(SiCH₂CH₂CH₂)₃P] and Ph₃CBr was substituted with Br-CEt₃ to prepare the target product.

### Example 5

A method for preparing the bulky and small-steric-hindrance ligand (PC₉H₁₈Si)CPh₃ adopted a lithiation method, also known as the Wurz method. The synthetic route in Example 1 was employed, and HC(CH═CH₂)₃ was substituted with HSi(CH₂CH═CH₂)₃ to prepare the target product.

The free radical addition method was the same as that in Example 2, and 1,4-pentadiene was substituted with 1,8-nonadiene to synthesize 10-phosphamonocyclodecane [(C₉H₁₈P)—H], and the target product was prepared, with a yield of 70%.

P(CH₂CH₂CH₂)₃Si—R (where R=CEt₃, CPr₃, or C(C₆H₁₁)₃) was synthesized by substituting Ph₃CBr with Et₃CBr, Pr₃CBr, or (C₆H₁₁)₃CBr, respectively.

### Example 6

A method for preparing the bulky and small-steric-hindrance ligand H{C[(CH₂)ₙ]₃P} (where n=3-7) was provided:

When n=3, HC[(CH₂)₃]₃P was synthesized by substituting HC(CH═CH₂)₃ with HC[(CH₂)₃-2CH═CH₂]₃, following the method described in Example 1.

When n=4, 5, 6, or 7, the target product was synthesized by substituting HC[(CH₂)₃-2CH═CH₂]₃ with HC[(CH₂)ₙ-2CH═CH₂]₃ using the same procedure.

A method for preparing the bulky and small-steric-hindrance ligand N[(CH₂)ₙ]₃P was provided:

Referring to the method in Example 1, the target product was obtained by substituting HC[(CH₂)ₙ-2CH═CH₂]₃ with N[(CH₂)ₙ-2CH═CH₂]₃.

A method for preparing the bulky and small-steric-hindrance ligand HSi[(CH₂)ₙ]₃P was the same as that for H{C[(CH₂)ₙ]₃P}, with HC[(CH₂)ₙ-2CH═CH₂]₃ substituted by HSi[(CH₂)ₙ-2CH═CH₂]₃.

### Example 7

A method for preparing the bulky and small-steric-hindrance ligand 2,5-dimethyl-3,4-dipropyltetrahydrofuran was provided: 2,5-dimethyl-3,4-dibromotetrahydrofuran was used as the raw material and reacted with 1-bromopropane in diethyl ether solvent via a Grignard reaction to yield the target product, with a yield of 65%.

A method for synthesizing the bulky and small-steric-hindrance ligand 2,5-dimethyl-3,4-dipropyltetrahydrothiophene was the same as that for 2,5-dimethyl-3,4-dipropyltetrahydrofuran, except that 2,5-dimethyl-3,4-dibromotetrahydrothiophene was used as the raw material instead of 2,5-dimethyl-3,4-dibromotetrahydrofuran. The yield was 60%.

A method for preparing the bulky and small-steric-hindrance ligand 2,5-dimethyl-3,4-dipropylpyrrolidine was the same as that for 2,5-dimethyl-3,4-dipropyltetrahydrofuran, except that 2,5-dimethyl-3,4-dibromopyrrolidine was used as the raw material instead of 2,5-dimethyl-3,4-dibromotetrahydrofuran. The yield was 67%.

### Example 8

A method for preparing the bulky and small-steric-hindrance ligand 2,6-dimethyl-3,5-dipropyl-1-thiacyclohexane was provided: 2,6-dimethyl-3,5-dibromo-1-thiacyclohexane was used as the raw material and reacted with 1-bromopropane via the Grignard reagent method to yield the product, with a yield of 58%.

A method for preparing the bulky and small-steric-hindrance ligand 2,6-dimethyl-3,5-dipropyl-1-oxacyclohexane was the same as that for 2,6-dimethyl-3,5-dipropyl-1-thiacyclohexane, except that 2,6-dimethyl-3,5-dibromo-1-oxacyclohexane was used as the raw material instead of 2,6-dimethyl-3,5-dibromo-1-thiacyclohexane. The yield was 67%.

A method for preparing the bulky and small-steric-hindrance ligand 2,6-dimethyl-3,5-dipropyl-1-azacyclohexane (or hydrogenated pyridine) was the same as that for 2,6-dimethyl-3,5-dipropyl-1-thiacyclohexane, except that 2,6-dimethyl-3,5-dibromo-1-azacyclohexane was used as the raw material instead of 2,6-dimethyl-3,5-dibromo-1-thiacyclohexane. The yield was 65%.

### Example 9

A method for synthesizing the bulky and small-steric-hindrance ligand PPh₃-C₁₂H₁₄N was provided: 8-bromojulolidine (C₁₂H₁₄BrN) was used as the raw material and reacted with 1-bromotriphenylmethane via the Grignard reagent method to yield the product with a yield of 55%.

### Example 10

A method for preparing the bulky and small-steric-hindrance ligand 3,4-diethylaluminum-thiophene was provided: Under nitrogen protection, 60 mmol of triethylaluminum was dissolved in toluene, and 10 mmol of 3,4-dibromothiophene was added at room temperature. The mixture was stirred for 5 hours, and the reaction was terminated. Toluene and unreacted raw materials were distilled off under reduced pressure (0.3 mmHg, 70° C.), and the target product was collected from the bottom of the reactor, with a yield of 57%.

A method for synthesizing the bulky and small-steric-hindrance ligand 3,4-di-trimethylmethyl-thiophene was provided via the Grignard reagent method using 3,4-dibromothiophene and bromotrimethylmethane as raw materials.

### Example 11

A method for synthesizing the bulky and small-steric-hindrance ligand 4-triphenylmethyl-2,5-dimethyl-1,4-diazacyclohexane included the following steps:
Step 1: Synthesis of lithium 2,5-dimethyl-1,4-diazacyclohexane, following the method described in Example 1
Step 2: Reaction of lithium 2,5-dimethyl-1,4-diazacyclohexane with bromotriphenylmethane to obtain the product.

A method for synthesizing the bulky and small-steric-hindrance ligand 3,5-dipropyl-phenyl-piperazine was the same as that for 4-triphenylmethyl-2,5-dimethyl-1,4-diazacyclohexane, except that 1-bromotriphenylmethane was substituted with 1-bromo-3,5-dipropylbenzene.

### Example 12

A method for synthesizing the bulky and small-steric-hindrance ligand diethylaluminum-substituted thiazolidine (C₃H₆NS)—R was provided via the Wurz method as described in Example 1, using thiazolidine (C₃H₇NS) and triethylaluminum as raw materials.

### Example 13

A method for synthesizing the bulky and small-steric-hindrance ligand 4-triphenylmethyl-2,6-dimethyl-1-oxa-4-aza-monocyclohexane was provided via the lithiation method as described in Example 1, using 2,6-dimethyl-1-oxa-4-aza-monocyclohexane as the raw material.

### Example 14

A method for synthesizing the bulky and small-steric-hindrance ligand C₁₆H₉—(PC₅H₁₀) was provided via the Wurtz method as described in Example 1, where C₅H₁₀P—Li reacted with C₁₆H₉Br (1-bromopyrene) to form the product.

Bulky and small-steric-hindrance ligands containing pyrene (C₁₆H₉) substituents, such as C₁₆H₉—(PC₄H₈), C₁₆H₉—PPh₂, C₁₆H₉—(PC₄H₄), and C₁₆H₉—(PO₂C₂H₄), could be synthesized via the Grignard reagent method as described in Example 7.

For example, C₁₆H₉—(PC₄H₈) was synthesized via the Grignard reagent method using C₁₆H₉MgBr and C₄H₈PBr as raw materials, with a yield of 50%.

A method for synthesizing bulky and small-steric-hindrance ligands C₁₀H₁₅—(PC₅H₁₀), C₁₀H₁₅—(PC₄H₄), C₁₀H₁₅—(PO₂C₂H₄) or C₁₈H₁₁—PPh₂, C₁₈H₁₁—(PC₄H₈), C₁₈H₁₁—(PC₅H₁₀), C₁₈H₁₁—(PC₄H₄), C₁₈H₁₁—(PO₂C₂H₄) was the same as that for the synthesis of C₁₆H₉—(PC₄H₈), C₁₆H₉—PPh₂, C₁₆H₉—(PC₄H₄), C₁₆H₉—(PO₂C₂H₄), with C₁₆H₉ substituted with C₁₀H₁₅ or C₁₈H₁₁, respectively.

### Example 15

A synthetic route for the bulky and small-steric-hindrance ligand phenyl-10-phosphamonocyclodecane [(C₉H₁₈P)-Ph] was as follows:

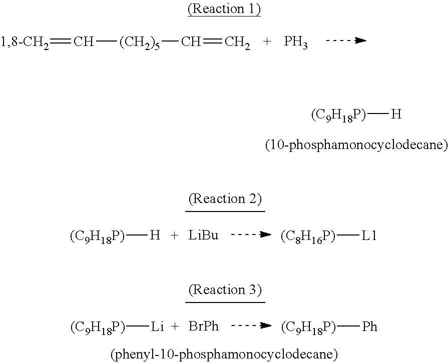

Synthesis methods: Reaction 1 was carried out by a free radical addition method; Reactions 2 and 3 were carried out by the Wurz method, using (C₉H₁₈P)—H and BrPh as raw materials. The target product was synthesized following the procedure described in Example 1. The synthesis method for C₁₁H₂₂PH was the same as above.

### Example 16

A method for synthesizing the bulky and small-steric-hindrance ligand (C₁₁H₂₂P)—NR included the following steps:
Step 1: C₁₁H₂₂PH was synthesized according to the preparation method described in Example 15;
Step 2: C₁₁H₂₂PH was converted to C₁₁H₂₂PCl by chlorination;
Step 3: C₁₁H₂₂P—NC₅H₁₀ was synthesized by dehydrochlorination.

Procedure: 10 mmol of (C₁₁H₂₂P)Cl was dissolved in 10 mL of MeCN. A mixed solution of 15 mmol of C₅H₁₀NH and 24 mmol of NEt₃ in 10 mL of MeCN was added dropwise within 5 minutes under stirring at room temperature. The mixture was stirred at room temperature for 16 hours, and the solvent was removed under reduced pressure to obtain a white solid. Ammonium chloride salt was removed by extraction with diethyl ether. The yield of the target product [(C₉H₁₈P)—C₅H₁₀N] was 50%.

### Example 17

A method for preparing the bulky and small-steric-hindrance ligand C₁₄H₁₂N—(PC₄H₈) was provided via the dehydrochlorination method as described in Example 16, using (C₄H₈P)Cl and H(NC₁₄H₁₂) as raw materials. The yield was 50%.

### Example 18

A method for synthesizing the bulky and small-steric-hindrance ligand (C₅H₁₀P)—(CH₂)ₙ—(C₅H₁₀) (where n=3-9) was provided via free radical addition. For example, the synthesis of the long-bridged bidentate ligand (C₅H₁₀P)—(CH₂)₅—(C₅H₁₀) was as follows:

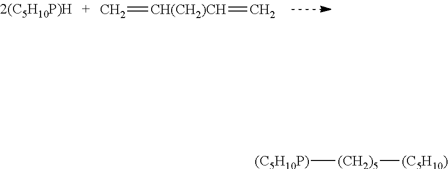

Procedure: Under the protection of high-purity nitrogen, 6.8 g (0.1 mol) of 1,4-pentadiene was weighed and dissolved in 100 mL of toluene. The solution was transferred to a 200 mL autoclave equipped with magnetic stirring, then 0.01 mol of AIBN initiator and 0.21 mmol of 6-phosphinocyclohexylphosphine (C₅H₁₀PH) were added. High-purity nitrogen was charged to 0.3 MPa, and stirring was initiated. The reaction temperature was controlled at 70-80° C. After 3 hours of reaction, an additional 0.01 mol of AIBN initiator was added, and the reaction was continued for 2 hours before termination. The solvent and unreacted materials were distilled off under reduced pressure, and the residue at the bottom of the reactor was collected to obtain the product. The content was analyzed by chromatography to confirm the formation of the long-chain diphosphine compound. The yield was 57% (based on 1,4-pentadiene).

### Example 19

A method for synthesizing the bulky and small-steric-hindrance ligand (C₅H₁₀P)NMe(CH₂)₇NMe(PC₅H₁₀) was provided via the dehydrochlorination method:

Using (C₅H₁₀P)Cl and HNMe(CH₂)₇NMeH as raw materials, the product was synthesized following the procedure described in Example 16, with a yield of 57%.

By substituting HNMe(CH₂)₇NMeH (10 mmol) with (H₂N(CH₂)O(CH₂)O(CH₂)NH₂, H₂N—C₆H₄—CH₂CH₂—C₆H₄—NH₂ or H₂N—(CH₂)₃—NH—(CH₂)₃—NH₂, ligands such as (C₄H₄P)—(C₁₄H₁₄N₂)—(PC₄H₄), (C₅H₁₀P)—(C₁₄H₁₄N₂)—(PC₅H₁₀), (C₈H₁₄P)—(C₁₄H₁₄N₂)—(PC₈H₁₄) or (C₄H₄P)—(C₆H₁₅N₃)—(PC₄H₄), (C₅H₁₀P)—(C₆H₁₅N₃)—(PC₅H₁₀), (C₂H₄O₂P)—(C₆H₁₅N₃)—(PO₂C₂H₄), (C₈H₁₄P)—(C₆H₁₅N₃)—(PC₈H₁₄) could be synthesized.

### Example 20

A method for synthesizing the bulky and small-steric-hindrance ligand Ph₂P—NC₅H₉-2-C₁₃H₇-7-C₅H₉N—PPh₂ was provided.

Preparation of HNC₅H₉—C₁₃H₇—C₅H₉NH: Synthesized via the Grignard reagent method using BrMg—C₁₃H₇—MgBr and 3-BrC₅H₉N (3-bromo-azacyclohexane) as raw materials. The yield of HNC₅H₉—C₁₃H₇—C₅H₉NH was 55%.

Preparation of Ph₂P—NC₅H₉-2-C₁₃H₇-7-C₅H₉N—PPh₂: Synthesized via the Wurtz method using HNC₅H₉—C₁₃H₇—C₅H₉NH and Cl—PPh₂ as raw materials. The yield of the target product was 57%.

A method for preparing the bulky and small-steric-hindrance ligand Ph₂P—(NC₅H₉)—C₁₃H₇—(PC₅H₉)—PPh₂ was the same as that for Ph₂P—(NC₂₃H₂₅N)—PPh₂, except that 2,7-di-4-bromopiperidinofluorene was used instead of 2,7-di-3-bromo-piperidine. The yield was 55%.

### Example 21

A method for preparing a bulky and small-steric-hindrance ligand was provided via the dehydrochlorination method as described in Example 16, using 2-HNMe-C₁₃H₈-7-MeNH and Cl—PC₈H₁₄ as raw materials.

### Example 22

A method for synthesizing the bulky and small-steric-hindrance ligand Ph₂P—N(CH₂CH₂)₂N—C₁₃H₇—N(CH₂CH₂)₂N—PPh₂ was provided via the dehydrobromination method, using HN(CH₂CH₂)₂N—C₁₃H₈—N(CH₂CH₂)₂NH and Br—PPh₂- as raw materials.

### Example 23

A method for synthesizing the bulky and small-steric-hindrance ligand Ph₂P—N(CH₂CH₂)₂N—C₁₃H₇—N(CH₂CH₂)₂N—PPh₂ was the same as that in Example 22, except that Br-Ph-Cl₃H₇-Ph-Br was used instead of 2,7-dibromofluorene (Br—C₁₃H₇—Br).

### Example 24

Bulky and small-steric-hindrance ligands No. 48 and No. 49 were synthesized via the Grignard reagent method following the procedure described in Example 7.

### Example 25

A method for preparing the bulky and small-steric-hindrance ligand Ph₂P—C₆H₄—C₆H₄—(C₈H₄S₂)—C₆H₄—C₆H₄—PPh₂ was provided via the Grignard reagent method, using 5,5′-dibromobithiophene as the raw material.

### Example 26

A method for synthesizing the bulky and small-steric-hindrance ligand (C₄H₈P)—(NC₄H₄)-Ph-C₁₃H₇-Ph-(C₄H₄N)—(PC₄H₈) was provided via the dehydrochlorination method as described in Example 16, using H(NC₄H₄)-Ph-C₁₃H₇·Ph-(C₄H₄N)H and Cl—(PC₄H₈) as raw materials.

### Example 27

A method for synthesizing the bulky and small-steric-hindrance ligand CP—(NC₅H₁₀)—C₆H₄—C₁₃H₈—C₆H₄—C₆H₄-E (where E=2,5-di-Me-C₄H₄S) included the following steps:
Step 1, (C₅H₁₀P)—(NC₅H₁₀)—C₆H₄—C₁₃H₈—C₆H₄—C₆H₄—Br was synthesized via the dehydrobromination method as described in Example 22, using H—(NC₅H₁₀)—C₆H₄—C₁₃H₈—C₆H₄—C₆H₄—Br and Cl—(PC₅H₁₀) as raw materials.
Step 2, (C₅H₁₀P)—(NC₅H₁₀)—C₆H₄—C₁₃H₈—C₆H₄—C₆H₄—(C₄H₄S) was synthesized via the Grignard reagent method using (C₅H₁₀P)—(NC₅H₁₀)—C₆H₄—C₁₃H₈—C₆H₄—C₆H₄—Br and 3-Br—(C₄H₄S) as raw materials.

Target products with other substituents for CP or E could be synthesized using the same method.

### Example 28

A method for preparing the bulky and small-steric-hindrance ligand (2.6-Me[O(C₂H₄)₂N]—C₆H₄—C₆H₄—C₁₃H₈—C₆H₄—C₆H₄—[C(PCH₂CH₂)₃ was provided via the dehydrobromination method as described in Example 16, using Br—C₆H₄—C₆H₄—C₁₃H₇—C₆H₄—C₆H₄—[PC(CH₂CH₂)₃] and (2.6-Me[O(C₂H₄)₂N]H as raw materials. Target products with other groups for E could be synthesized using the same method.

### Example 29

A method for synthesizing the bulky and small-steric-hindrance ligand PPh₂-C₆H₄-Ph-C₁₃H₇-Ph-C₆H₄—PPh₂ was provided via the Grignard method, using 3-BrMg—C₆H₄-Ph-C₁₃H₇-Ph-C₆H₄-4-MgBr and Br—PPh₂ as raw materials.

### Example 30

A method for synthesizing the bulky and small-steric-hindrance ligand [N(CH₂CH₂)₃C]—C₆H₄—C₆H₄—C₁₃H₇—C₆H₄—C₆H₄—[N—(CH₂CH)₂O] was provided via the dehydrobromination method, using [N(CH₂CH₂)₃C]—C₆H₄—C₆H₄—C₁₃H₈—C₆H₄—C₆H₄—Br and H—[N—(CH₂CH)₂O] as raw materials. Target products with other groups for E₁ and E₂ could be synthesized using the same method.

### Example 31

A method for synthesizing the bulky and small-steric-hindrance ligand N—(CH₂CH₂)₃C]—C₆H₄—C₁₃H₇—C₆H₄—[C—(CH₂CH₂)₃N was provided via the Grignard reagent method, using BrMg—C₆H₄—C₁₃H₇—C₆H₄—MgBr and Br—[C—(CH₂CH₂)₃N] as raw materials. The yield was 50%.

### Example 32

A ternary catalytic system composed of a bulky and small-steric-hindrance ligand, a central metal, and an activator (ligand/transition metal/activator) was used for ethylene oligomerization to produce 1-octene and 1-hexene.

### Procedure:

A 500 mL stainless steel autoclave was cleaned and vacuum-dried at 120° C. for 3 hours, then cooled to room temperature. In a glovebox, the chromium source (Cr), organophosphine ligand (L), and activator (A) were separately dissolved in a specified amount of dry methylcyclohexane solvent according to the molar ratio required by the experiment. The solutions were stored in 100 mL syringes and sealed with silicone rubber. At room temperature, the chromium source (Cr) and organophosphine ligand (L) were first injected into the autoclave. Ethylene was immediately charged to 0.3 MPa, and the system was purged by stirring three times. The oxygen content in the ethylene gas phase inside the autoclave was detected using a micro-oxygen analyzer, which was required to be below 3 ppm. After passing the test, the activator was added via a pressure bomb under an ethylene atmosphere. Ethylene was immediately pressurized to 2.5 MPa, and the temperature was rapidly raised to the designed temperature (40-80° C.). The reaction pressure was controlled at 5.0 MPa with a stirring speed of >500 rpm. The reaction was stopped after 30 minutes. The autoclave was cooled to 0° C. The exhausted gas phase was captured, and an appropriate amount of isooctanol or 30 mL of 10% HCl was added to quench the activator. Liquid samples were taken for chromatographic analysis, the products were weighed, and the filtered byproduct PE was dried and weighed. The yields of gas, liquid, and solid products, catalytic efficiency (kg of oligomer product/g of Cr), product distribution, and purities of 1-octene, 1-hexene, and 1-butene were calculated.

Chromium source materials could be selected individually from CrCl₃(THF)₃, CrCl₂(THF)₂, Cr(acac)₃, and Cr(HA)₃.

Activators (A) could be selected individually from (1) methylaluminoxanes (MAO or MMAO-3A), (2) alkylaluminum compounds (triethylaluminum or triisobutylaluminum), and (3) DMAO.

Alternatively, a mixture of (1)+(3) could be used.

Reaction conditions: Al/Cr molar ratio=100-500; L/Cr molar ratio=1-1.5:1; chromium concentration=5 μmol.

The evaluation results are provided in Table 3.

TABLE 3
Evaluation Results of Ethylene Oligomerization of Chromium-Based
Ternary Catalysts with Bulky and Small-Steric-Hindrance Ligands

| | | | | Catalytic | | | | | |
|---|---|---|---|---|---|---|---|---|---|
| | Total | Ligand Molecular | Time | Efficiency | | | | | PE |
| No. | No. | Formula | (min) | Kg · (gCr)⁻¹ | C4% | C6% | C8% | C10+ % | % |
| 1 | 5 | P[(—CH₂)ₙ—]₃CR | 30 | 377 | 12.5 | 9.1 | 75.7 | 0 | 2.7 |
| | | n = 5, R—Me | | | (99) | (98) | (97) | | |
| 2 | 37 | R—(PC₁₀H₂₀) | 30 | 452 | 17.7 | 13.0 | 67.5 | 0 | 1.8 |
| | | | | | (99) | (98) | (97) | | |
| 3 | 41 | Ph₂P—NH—Ph—CH₂CH₂—Ph—NH—PPh₂ | 30 | 170 | 30.7 | 16.1 | 52.5 | 0 | 0.7 |
| | | | | | (99) | (98) | (97) | | |
| 4 | 43 | H₁₀C₅P—NC₅H₉—C₁₃H₈—C₅H₉N—PC5H₁₀ | 30 | 307 | 10.3 | 9.8 | 79.7 | 0 | 0.1 |
| | | | | | (99) | (98) | (97) | | |
| 5 | 48 | Ph₂P—C₆H₄—C₁₃H₈—C₆H₄—PPh₂ | 30 | 300 | 34.0 | 7.7 | 58.7 | 0 | 0.1 |
| | | | | | (99) | (98) | (97) | | |
| 6 | 60 | NH₁₂C₇—C₆H₄—C₁₃H₈—C₆H₄—PC₇H₁₂ | 30 | 401 | 7.5 | 6.6 | 85.7 | 0 | 0.2 |
| | | | | | (99) | (98) | (97) | | |

Reaction conditions were as follows: Temperature: 80° C.; Pressure: 3.0-4.0 MPa; Al/Cr ratio=500 (mol); Catalyst concentration=5 μmol. Data in parentheses represented the linear terminal olefin ratio of the product (%).

The basic principles, main features and advantages of the present disclosure have been shown and described above. It should be understood by those skilled in the art that the present disclosure is not limited to the foregoing embodiments, and the embodiments and descriptions set forth above are merely preferred examples of the present disclosure and are not intended to limit the present disclosure. Without departing from the spirit and scope of the present disclosure, various changes and improvements can be made thereto, and all such changes and improvements shall fall within the scope of the present disclosure as claimed. The scope of protection claimed by the present disclosure is defined by the appended claims and equivalents thereof.

1. A bulky and small-steric-hindrance ligand, wherein the bulky and small-steric-hindrance ligand is a monodentate bulky and small-steric-hindrance ligand constructed from dentate groups consisting of dentate elements without substituents, a monodentate bulky and small-steric-hindrance ligand constructed from dentate groups consisting of dentate elements containing branched substituents, a bidentate ligand composed of two dentate groups of P or N linked by a bridging group, or a bidentate ligand composed of two identical or different dentate groups containing one or more dentate elements selected from P, N, S, and O linked by a bridging group that does not change configuration due to σ-rotation, the bridging group has a double-bonded conjugated system; the dentate elements are one or more of P, N, S, and O.

2. The bulky and small-steric-hindrance ligand according to claim 1, wherein the monodentate bulky and small-steric-hindrance ligand constructed from dentate groups consisting of dentate elements containing branched substituents is specifically one or more of the following: a monodentate tertiary phosphine ligand compound containing only one dentate group, a monodentate tertiary amine ligand compound containing only one dentate group, a monodentate thioheterocyclic ligand compound containing only one dentate group, and a monodentate oxoheterocyclic ligand compounds containing only one dentate group; the bidentate ligand composed of two identical or different dentate groups containing one or more dentate elements selected from P, N, S, and O linked by a bridging group that does not change configuration due too-rotation is a bidentate ligand compound composed of two dentate groups linked by a bridging group, wherein the two dentate groups of the bidentate ligand are located at two coordination sites that can form a straight line with the central metal, and the ligand and the central metal form an arch-shaped structure, providing sufficient operational space for the self-assembly of the four π-ethylene groups located at the central metal into the target product 1-octene.

3. The bulky and small-steric-hindrance ligand according to claim 1, wherein the monodentate bulky and small-steric-hindrance ligand constructed from dentate groups consisting of dentate elements without substituents is as follows:

| Serial | | Skeletal | |
|---|---|---|---|
| No. | Ligand | Formula | Substituents and Coefficients |
| 1 | 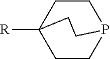 | C₇H₁₂P | R = Cph₃, CEt₃, (CH₂)nCH₃, Ph(CH₂)nCH₃, C₆H₁₁(CH₂)nCH₃, PhCOOR |
| 2 | 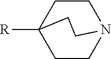 | C₇H₁₂N | R = Cph₃, CEt₃, (CH₂)nCH₃, Ph(CH₂)nCH₃, C₆H₁₁(CH₂)nCH₃ |
| 3 | 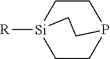 | C₆H₁₂PSi | R = Cph₃, (CH₂)nCH₃, CEt₃, Ph(CH₂)nCH₃ |
| 4 | 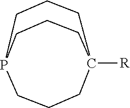 | C₁₀H₁₈P | R = Cph₃, (CH₂)nCH₃, CEt₃, Ph(CH₂)nCH₃ |
| 5 | 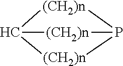 | — | n = 3, 4, 5, 6, 7 |
| 6 | 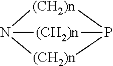 | — | n = 3, 4, 5, 6, 7 |
| 7 | 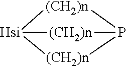 | — | n = 3, 4, 5, 6, 7 |
| 8 | 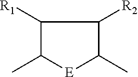 | C₁₀H₁₈ | R₁ = R₂ = Et, Pr E = S, O |
| 9 | 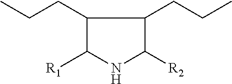 | C₁₀H₁₉N | R₁ = R₂ = H, Me |
| 10 | 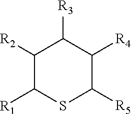 | C₅H₅S | R₁ = R₅ = H, Me R₂ = R₄ = H, Et, Pr, t-Bu R₃ = H, Pr, Cph₃, AlEt₂ |
| 11 | 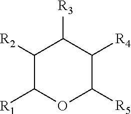 | C₅H₅O | R₁ = R₅ = H, Me R₂ = R₄ = H, Et, Pr, t-Bu R₃ = H, Pr, t-Bu, Cph₃ |
| 12 | 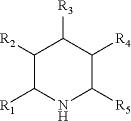 | C₅H₆N | R₁ = R₅ = H, Me R₂ = R₄ = H, Et, Pr, t-Bu R₃ = H, Pr, t-Bu, Cph₃ |
| 13 | 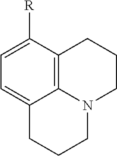 | C₁₂H₁₄N | R = Cph₃, CEt₃, CMe₃ |
| 14 | 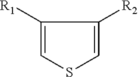 | C₄H₂S | R₁ = R₂ = Cph₃, CMe₃, CEt₃, Cpr₃, AlEt₂, Al(iBu)₂ |
| 15 | 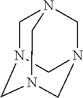 | C₆H₁₂N₄ | — |
| 16 | 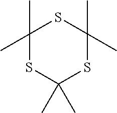 | C₉H₁₈S₃ | — |
| 17 | 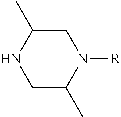 | C₆H₁₃N₂ | R = cph₃, CEt₃, AlEt₂, Al(iBu)₂, C(t-Bu)₃ |
| 18 | 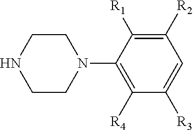 | C₁₁H₁₂N₂ | R₁ = R₄ = H, Me R₃ = R₂ = Pr t-Bu |
| 19 | 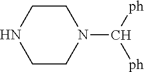 | C₁₇H₂₀N₂ | — |
| 20 | 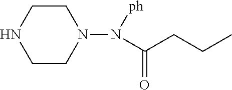 | C₁₄H₂₀N₂O | — |
| 21 | 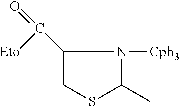 | C₅H₇O₂NS | — |
| 22 | 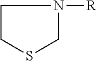 | C₃H₆NS | R = AlEt₂, Ali-Bu₃, cph₃, cpr₃ |
| 23 | 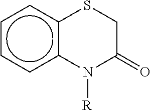 | C₈H₆NOS | R = cph₃, CEt₃, cpr₃, C(t-Bu)₃ |
| 24 | 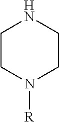 | C₅H₁₁N₂ | R = cph₃, CEt₃, AlEt₂, Al(i-Bu)₂ |
| 25 | 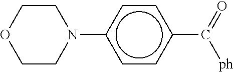 | C₁₇H₁₇NO₂ | — |
| 26 | 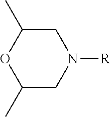 | C₆H₁₂NO | R = cph₃, CEt₃, Cpr₃, C(t-Bu)₃, Al(Bu)₃ |
| 27 | 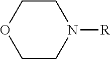 | C₄H₈NO | R = cph₃, CEt₃, Cpr₃, C(t-Bu)₃, Al(Bu)₃ |
| 28 | 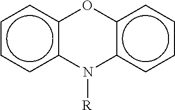 | C₁₂H₈NO | R = cph₃, CEt₃, AlEt₂, Cpr₃, C(t-Bu)₃, Al(i-Bu)₂ |
| 29 | 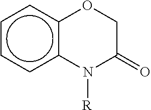 | C₈H₆NO₂ | R = cph₃, CEt₃, AlEt₂, Cpr₃, C(t-Bu)₃, Al(i-Bu)₂ |
| 30 | 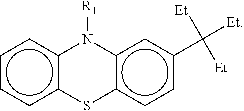 | C₁₉H₂₂NS | R = cph₃, AlEt₂, CEt₃ |

4. The bulky and small-steric-hindrance ligand according to claim 1, wherein the monodentate bulky and small-steric-hindrance ligand constructed from dentate groups consisting of dentate elements containing branched substituents is as follows:

| Serial | | Skeletal | | | | |
|---|---|---|---|---|---|---|
| No. | Ligand | Formula | Substituents and Coefficients | | | |
| 31 | 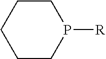 | PC₅H₁₀ | 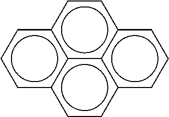 | 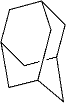 | 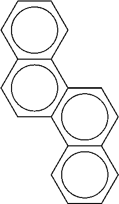 | 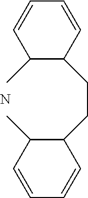 |
| 32 | 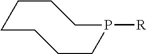 | PC₇H₁₄ | 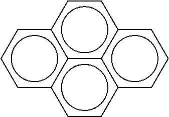 | 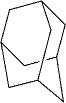 | 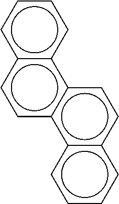 | 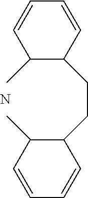 |
| 33 | 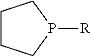 | PC₄H₈ | 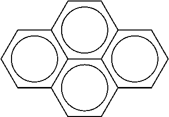 | 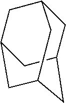 | 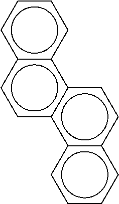 | 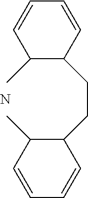 |
| 34 | 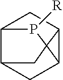 | PC₇H₁₀ | 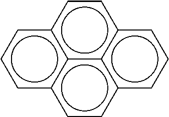 | 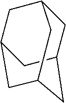 | 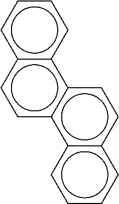 | 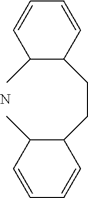 |
| 35 | 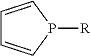 | PC₄H₄ | 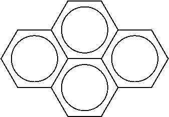 | 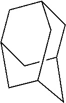 | 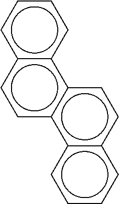 | 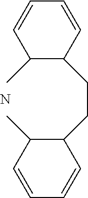 |
| | | | | | | |
|---|---|---|---|---|---|---|
| 36 | 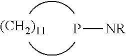 | PC₁₁H₂₂ | NR = 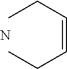 | 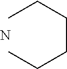 | 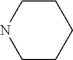 | 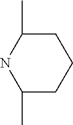 |
| | | |
|---|---|---|
| 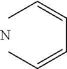 | 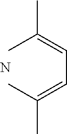 | 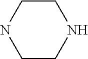 |
| | | | |
|---|---|---|---|
| 37 | 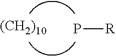 | PC₁₀H₂₀ | R = Me, Et, Pr, ph, C₄H₁₁ = 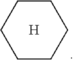 |

5. The bulky and small-steric-hindrance ligand according to claim 1, wherein the bidentate ligand composed of two dentate groups of P or N linked by a bridging group is:

| Seri- | | | | | |
|---|---|---|---|---|---|
| al | | | Dental Group and | | |
| No. | Ligand | Skeletal Formula | Coefficient | | |
| 38 | CP₁—(CH₂)n—CP₂ | —(CH₂)n— | cp₁ = 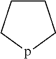 | 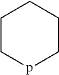 | pph₂ |
| | | | cp₂ = 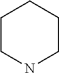 | 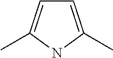 | |
| | | | n = 3, 5, 7, 9, 4 | | |
| | | | cp₁ = cp₂ or cp [symbol] ≠ cp₂ | | |
| 39 | CP—N(Me)—(CH₂)ₙ—N(Me)—CP | —(Me)N—(CH₂)ₙN(Me)— | CP = 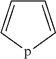 | 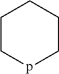 | 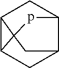 |
| | | | pph₂ | 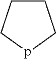 | C₇H₁₀P |
| | | | n = 3, 4, 5, 6, 7 | | |
| 40 | CPNH(CH₂)O(CH₂)O(CH₂)NHCP | C₈H₁₈N₂O₂ | CP = 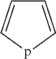 | 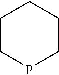 | 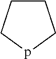 |
| | |
|---|---|
| 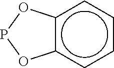 | 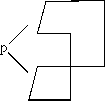 |
| | | | pph₂ | | |
|---|---|---|---|---|---|
| 41 | 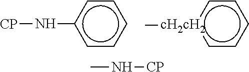 | C₁₄H₁₄N₂ | CP = 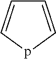 | 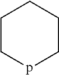 | 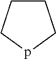 |
| | |
|---|---|
| 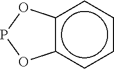 | 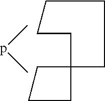 |
| | | | pph₂ | | |
|---|---|---|---|---|---|
| 42 | CP—NH—(CH₂)₃—NH—(CH₂)₃—NH—CP | C₆H₁₅N₃ | CP = 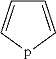 | 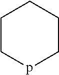 | 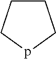 |
| | |
|---|---|
| 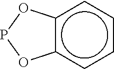 | 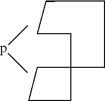 |

pph₂ [symbol]
indicates data missing or illegible when filed

6. The bulky and small-steric-hindrance ligand according to claim 1, wherein the bidentate ligand composed of two identical or different dentate groups containing one or more dentate elements selected from P, N, S, and O linked by a bridging group that does not change configuration due to σ-rotation is:

| Serial | | Skeletal | | | |
|---|---|---|---|---|---|
| No. | Ligand | Formula | Dental Group and Coefficient | | |
| 43 | 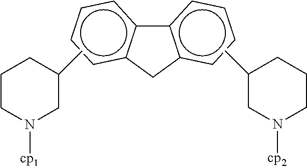 | C₂₃H₂₆N₂ | cp₁ = cp₂ | 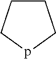 | 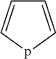 |
| | | | 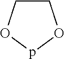 pph₂ | 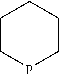 | 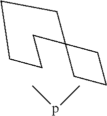 |
| 44 | 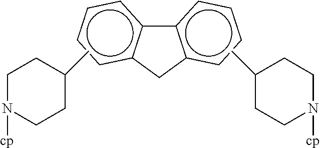 | C₂₃H₂₆N₂ | cp₁ = cp₂ 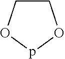 pph₂ | 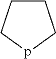 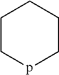 | 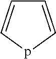 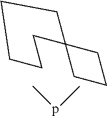 |
| 45 | 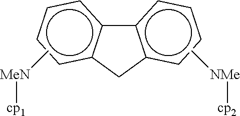 | C₁₅H₁₄N₂ | cp₁ = cp₂ 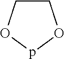 pph₂ | 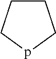 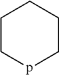 | 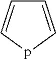 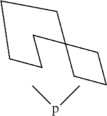 |
| 46 | 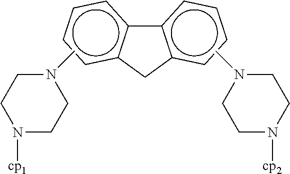 | C₂₁H₂₄N₄ | cp₁ = cp₂ 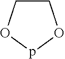 pph₂ | 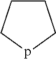 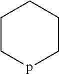 | 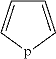 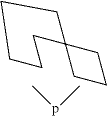 |
| 47 | 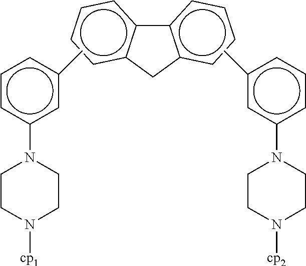 | C₃₃H₃₂N₄ | cp₁ = cp₂ 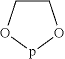 pph₂ | 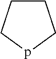 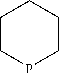 | 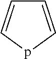 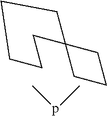 |
| 48 | 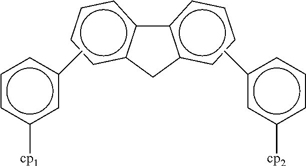 | C₂₅H₁₅ | cp [symbol] ≠ cp [symbol] cp [symbol] = cp [symbol] = 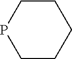 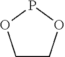 | 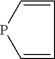 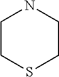 pph [symbol] = | 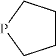 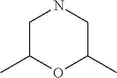 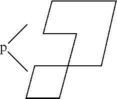 |
| | | | 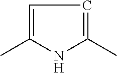 | 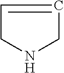 | 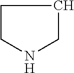 |
| | | | | 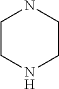 | |
| 49 | 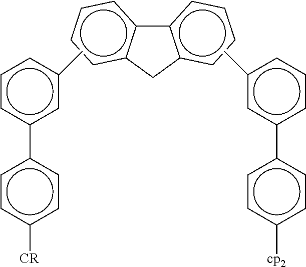 | C₃₇H₂₄ | cp [symbol] ≠ cp [symbol] cp [symbol] = cp [symbol] = 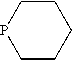 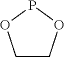 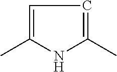 | 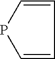 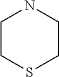 pph [symbol] 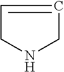 | 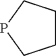 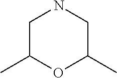 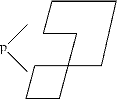 |
| | |
|---|---|
| 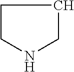 | 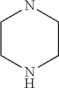 |
| | | | | | |
|---|---|---|---|---|---|
| 50 | 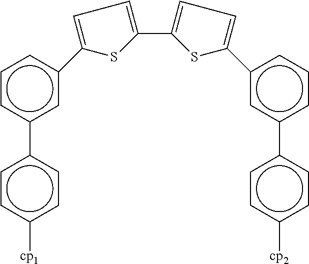 | C₃₂H₂₀S₂ | cp [symbol] ≠ cp [symbol] cp [symbol] = cp [symbol] = 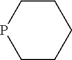 pph [symbol] 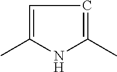 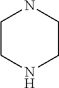 | 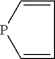 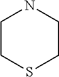 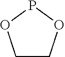 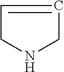 | 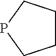 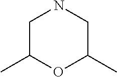 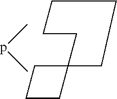 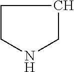 |
| | | | | | |
|---|---|---|---|---|---|
| 51 | 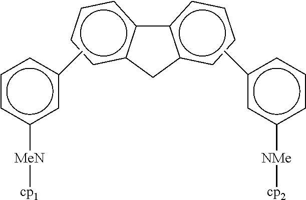 | C₂₇H₂₂N₂ | 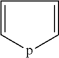 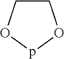 pph₂ | 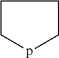 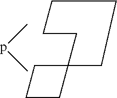 | 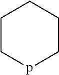 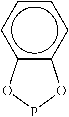 |
| 52 | 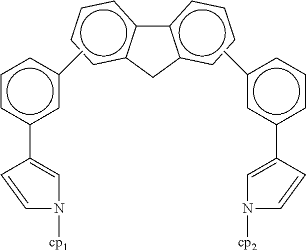 | C₃₃H₁₃N₂ | cp₁ = cp₂ 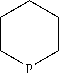 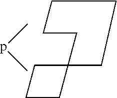 | 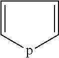 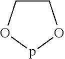 pph₂ | 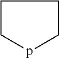 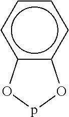 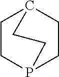 |
| | | | | | |
|---|---|---|---|---|---|
| 53 | 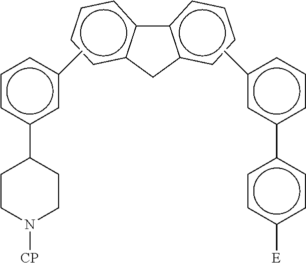 | C₃₁H₂₉N | cp = 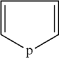 E = 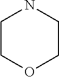 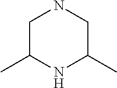 | pph₂ 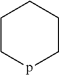 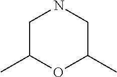 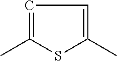 | 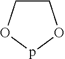 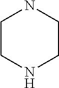 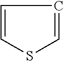 |
| | |
|---|---|
| 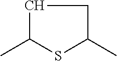 | 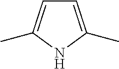 |
| | | | | |
|---|---|---|---|---|
| 54 | 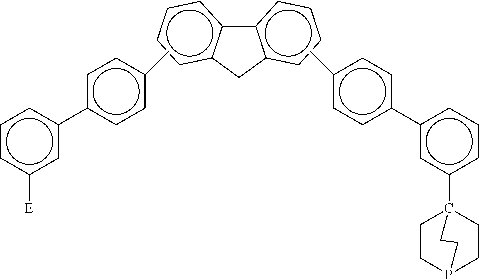 | C₄₄H₃₆P | E = 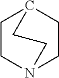 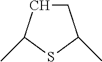 | 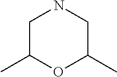 |
| 55 | 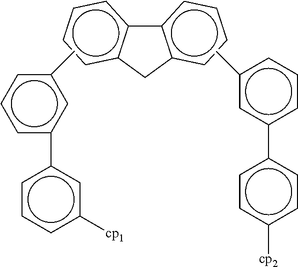 | C₃₇H₂₄ | cp1 = cp₂ = 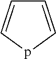 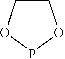 pph₂ | 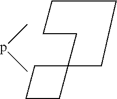 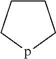 |
| 56 | 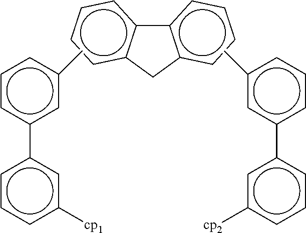 | C₃₇H₂₄ | cp₁ = cp₂ = 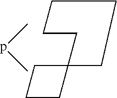 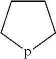 | 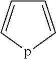 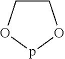 pph₂ |
| 57 | 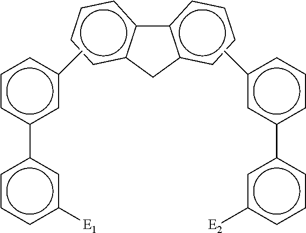 | C₃₇H₂₄ | E₁ = E₂ 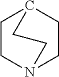 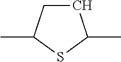 | E₁ ≠ E₂ 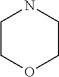 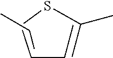 pph₂ |
| | | | |
|---|---|---|---|
| 58 | 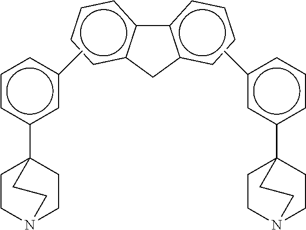 | C₂₇H₃₃N₂ | — |
| 59 | 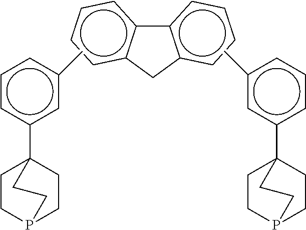 | C₂₇H₃₃P₂ | — |
| 60 | 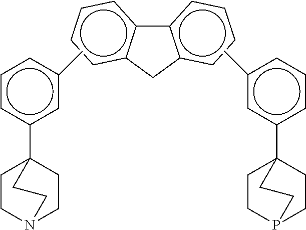 | C₂₇H₃₃NP | — |

[symbol]

indicates data missing or illegible when filed
the bridging group has a double-bonded conjugated system.

7. The bulky and small-steric-hindrance ligand according to claim 1, wherein the dentate groups are specifically as follows:
Dentate group 1, molecular formula: PPh₂;
Dentate group 2, molecular formula: C₈H₁₄P, structural formula: 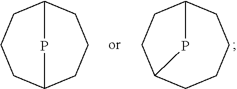
Dentate group 3, molecular formula: PC₄H₈;
Dentate group 4, molecular formula: PC₅H₁₀, structural formula: 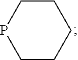
Dentate group 5, molecular formula: PC₆H₁₀O₂, structural formula: 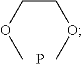
Dentate group 6, molecular formula: PC₇H₁₂, structural formula: 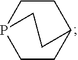
Dentate group 7, molecular formula: NC₇H₁₂, structural formula: 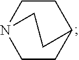
Dentate group 8, molecular formula: NC₄H₈S, structural formula: 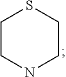
Dentate group 9, molecular formula: NC₄H₄;
Dentate group 10, molecular formula: NC₄H₆;
Dentate group 11, molecular formula: N₂C₄H₉, structural formula:

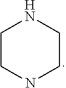

8. A ternary catalytic system, wherein the ternary catalytic system is a ligand/transition metal/activator ternary catalytic system comprising the bulky and small-steric-hindrance ligand of claim 1, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

9. The system according to claim 8, wherein the activator is an aluminum-containing activator, and the ternary catalytic system catalyzes the selective oligomerization of ethylene to produce three linear normal α-olefin products, generating only three linear normal α-olefin products of 1-octene, 1-hexene, and 1-butene, without generating C10-C20 waste olefins, and the arbon utilization rate is 100%.

10. The system according to claim 9, wherein the aluminum-containing activator is an alkylaluminum compound and/or an alkoxyaluminum compound, the alkylaluminum compound is specifically one or more of triethylaluminum, trimethylaluminum, and triisobutylaluminum, the alkoxyaluminum compound is specifically one or more of MAO, MMAO, DMAO, and MMAO-3A.

11. A ternary catalytic system, wherein the ternary catalytic system is a ligand/transition metal/activator ternary catalytic system comprising the bulky and small-steric-hindrance ligand of claim 2, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

12. A ternary catalytic system, wherein the ternary catalytic system is a ligand/transition metal/activator ternary catalytic system comprising the bulky and small-steric-hindrance ligand of claim 3, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

13. A ternary catalytic system, wherein the ternary catalytic system is a ligand/transition metal/activator ternary catalytic system comprising the bulky and small-steric-hindrance ligand of claim 4, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

14. A ternary catalytic system, wherein the ternary catalytic system is a ligand/transition metal/activator ternary catalytic system comprising the bulky and small-steric-hindrance ligand of claim 5, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

15. A ternary catalytic system, wherein the ternary catalytic system is a ligand/transition metal/activator ternary catalytic system comprising the bulky and small-steric-hindrance ligand of claim 6, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

16. A ternary catalytic system, wherein the ternary catalytic system is a ligand/transition metal/activator ternary catalytic system comprising the bulky and small-steric-hindrance ligand of claim 7, a central metal, and an activator, the central metal is one or more of Ti, Zr, V, Nb, Ta, Cr, Mo, W, Mn, Fe, Co, Ni, and transition metals of Group IVB, VB, and VIB of the periodic table.

17. The system according to claim 11, wherein the activator is an aluminum-containing activator, and the ternary catalytic system catalyzes the selective oligomerization of ethylene to produce three linear normal α-olefin products, generating only three linear normal α-olefin products of 1-octene, 1-hexene, and 1-butene, without generating C10-C20 waste olefins, and the arbon utilization rate is 100%.

18. The system according to claim 17, wherein the aluminum-containing activator is an alkylaluminum compound and/or an alkoxyaluminum compound, the alkylaluminum compound is specifically one or more of triethylaluminum, trimethylaluminum, and triisobutylaluminum, the alkoxyaluminum compound is specifically one or more of MAO, MMAO, DMAO, and MMAO-3A.

19. The system according to claim 12, wherein the activator is an aluminum-containing activator, and the ternary catalytic system catalyzes the selective oligomerization of ethylene to produce three linear normal α-olefin products, generating only three linear normal α-olefin products of 1-octene, 1-hexene, and 1-butene, without generating C10-C20 waste olefins, and the arbon utilization rate is 100%.

20. The system according to claim 19, wherein the aluminum-containing activator is an alkylaluminum compound and/or an alkoxyaluminum compound, the alkylaluminum compound is specifically one or more of triethylaluminum, trimethylaluminum, and triisobutylaluminum, the alkoxyaluminum compound is specifically one or more of MAO, MMAO, DMAO, and MMAO-3A.
